(19) United States
(12) Patent Application Publication
(10) Pub. No.: US 2005/0020580 A1
(43) Pub. Date: Jan. 27, 2005

(54) Materials and methods for the prevention or treatment of apoptosis and apoptosis-related diseases and conditions

(76) Inventors: Andrew D. Badley, Mantorville, MN (US); David H. Lynch, Bainbridge Island, WA (US)

Correspondence Address: QUARLES & BRADY LLP, 411 E. WISCONSIN AVENUE, SUITE 2040, MILWAUKEE, WI, 53202-4497, US

(21) Appl. No.: 10/741,493

(22) Filed: Dec. 19, 2003

Related U.S. Application Data
(60) Provisional application No. 60/436,114, filed on Dec. 23, 2002.

(51) Int. Cl. A61K 31/551; A61K 31/522

(52) U.S. Cl. 514/220; 514/263.36

(57) ABSTRACT

Compositions, kits and methods for reducing or inhibiting apoptosis in a cell or tissue and for treating or preventing an apoptosis-related disease or condition in a mammal that is not infected with HIV are disclosed. The methods involve administering to said mammal a therapeutically effective amount of one or more HIV protease inhibitors, and/or reducing mitochondrial adenine nucleotide translocator (ANT) dependent permeability transition pore complex (PTPC) opening and/or mitochondrial cytochrome c release in target cells or tissues.

### CROSS-REFERENCE TO RELATED APPLICATIONS

This application claims benefit to the U.S. provisional application Ser. No. 60/436,114, filed on Dec. 23, 2002, which is herein incorporated by reference in its entirety.

### STATEMENT REGARDING FEDERALLY SPONSORED RESEARCH OR DEVELOPMENT

--

### BACKGROUND OF THE INVENTION

There is compelling evidence that protease inhibitor (PI)-based antiretroviral therapy reduces lymphoid apoptosis in patients infected with HIV (Badley, A. D. et al, J. Clin. Invest. 102:79-87, 1998). It has also been shown that PI treatment of cells in vitro reduces apoptosis resulting from a diverse array of apoptotic stimuli including death receptor ligation, chemotherapeutic agents and growth factor withdrawal (Phenix et al., Apoptosis 7:295-312, 2002). While the exact mechanisms responsible for inhibition of apoptosis by PI remain unclear, the suggestion that PIs inhibit the mitochondrial permeability transition pore complex which is required for type II apoptotic signaling is supported by observations that: 1) PI inhibit forms of apoptosis that utilize a mitochondrial dependent (type II) pathway, but not a mitochondrial independent (type I) pathway; and 2) PIs block mitochondrial loss of transmembrane potential and consequent cytochrome-c release (Phenix et al., Blood 98:1078-1085, 2001).

The demonstration of enhanced survival and/or improved organ function following treatment with caspase inhibitors in models of ischemia/reperfusion injury (Endres et al, J Cereb. Blood Flow Metab. 18:238-247, 1998), burns (Fukuzuka et al., Ann. Surg. 229:851-859, 1999), endotoxemia (Grobmyer et al., Mol Med. 5:585-594, 1999), sepsis (Hotchkiss et al., Proc. Natl. Acad. Sci. USA 96:14541-14546, 1999), and sepsis following cecal ligation and perforation (CLP) (Hotchkiss et al., Nat. Immunol. 1:496-501, 2000) has led to the proposal that apoptosis is a significant factor in the morbidity and mortality associated with sepsis, and that antiapoptotic drugs would be beneficial in the therapy of sepsis (Oberholzer et al., Faseb J 15: 879-92, 2001).

Recent advances have implicated enhanced apoptosis in the pathogenesis of a broad cross section of human diseases or physiological insults including various forms of neurodegenerative diseases, autoimmune and inflammatory disorders, infectious diseases (such as from bacteria, viruses, protozoa, hepatitis, inflammatory bowel disease, etc.), ischemia/hypoperfusion, sepsis, ionizing and UV irradiation, chemotherapeutic agents and toxins, induce apoptosis in affected organs and tissues.

Accordingly, interest has shifted towards the development of a novel therapeutic approach using agents which safely modify the apoptotic response. Although HIV PI have been shown to inhibit apoptosis in vitro using experimental systems that require retroviral replication, it was previously not known whether these effects would apply clinically to apoptotic pathologies that do not involve retroviral infections.

Elucidation of the structure of an HIV protease as a homodimeric aspartyl protease facilitated the development of a class of peptidomimetic substrate inhibitors that have improved survival and reduced morbidity and mortality associated with HIV infection (Palella et al., N. Engl. J. Med. 338:853-860, 1998). HIV protease has been shown to cleave the HIV polyprotein into structural and functional proteins that are required for viral replication (Weiss, In: RNA Tumor Viruses, Vol. 2nd Rev. Edition Ed. (Cold Spring Harbor Laboratory, Cold Spring Harbor, N.Y., 1985). Thus, effective HIV therapy, which usually includes PI, dramatically reduces viral replication often resulting in undetectable levels of plasma viremia. Coincident with inhibited viral replication, there are quantitative and qualitative improvements in host immune function, including significant increases in both CD4 and CD8 T cell numbers and percentages (Autran et al., Science 277:112-116, 1997). The increase in CD4 T cell numbers that follows effective antiretroviral therapies is paralleled by reduced CD4 and CD8 T cell apoptosis in both peripheral blood and lymphoid tissues (Badley, A. D. et al., Cell Death Differ. 6:420-32, 1999; and Phenix et al., Apoptosis 7:295-312, 2002).

Many elements influence whether or not a cell will undergo apoptosis. Four cellular receptors are thus known to induce apoptosis after ligand binding, the Fas receptor, P55 tumor necrosis factor receptor (TNF-R), and TNF-related apoptosis-inducing ligand receptors (TRAIL) 1 and 2. Fas ligand, TNF, and TRAIL bind these receptors to initiate apoptosis. Upon ligand binding, these death receptors recruit the adaptor proteins FADD (Fas-associated death domain) and TRADD (TNF receptor-associated death domain) or both. In turn, these proteins sequentially activate a family of cysteine proteases, known as caspases, that cleave endogenous proteins at aspartate residues.

Cysteine-dependent aspartate-specific proteases (caspases) are a family of proteases that cleave their substrates at aspartic acid (D)-X bonds (where X is any amino acid). They are highly specific endopeptidases that catalyze limited proteolysis (Stennicke et al., Cell Death Differen. 6:1054-1059, 1999). To date 14 mammalian caspases have been identified.

Certain physical stimuli (e.g. chemotherapy, ultraviolet radiation, and ionizing radiation) can also cause apoptosis by inducing changes in the mitochondria that include opening of the permeability transition pore and loss of mitochondrial inner transmembrane potential. This mechanism allows the release of apoptosis regulatory proteins that promote further caspase activation and apoptosis. Fas-induced apoptosis has been shown to be mediated by either direct caspase activation with mitochondrial involvement (type 1) or a pathway that requires mitochondrial activation (type 2) or both.

Detectable reductions in apoptosis may occur before any significant antiretroviral effect is seen or may even occur in the absence of antiviral effect, suggesting that PIs modify apoptosis in a manner which is distinct from their effects on viral replication (Deeks et al., Aids 13:F35-43, 1999; Ledergerber et al., Lancet 353:863-868, 1999; Deeks et al., J. Infect. Dis. 181:946-953, 2000; Hawley-Foss et al, Clin. Infect. Dis 33:344-8, 2001). Despite these in vitro data, the significance of the antiapoptotic effects of PIs in vivo remains unknown.

To date, candidate apoptosis inhibitors have suffered from unacceptable toxicity, which limits their clinical utility. By contrast, the extensive clinical experience with HIV PIs indicates a satisfactory safety profile despite their unwanted long-term effects on lipid and glucose metabolism.

Thus, there is a need in the art to develop methods for inhibiting apoptosis in vivo Accordingly, it is an object of the present invention to provide methods for treating/preventing/inhibiting apoptotic pathologies in non-HIV infected cells using HIV PI.

### BRIEF SUMMARY OF THE INVENTION

The present invention relates to methods of preventing or treating apoptosis in a cell or tissue of a mammal that is not infected with HIV by administering to said mammal a therapeutically effective amount of one or more HIV protease inhibitors. Using HIV protease inhibitors nelfinavir (NFV) and ritonavir (RIT), and animal models for several diseases and conditions (sepsis and septic shock, hepatitis, stroke, toxin mediated diseases and conditions, amyotrophic lateral sclerosis, and multiple sclerosis) that involve the apoptosis of various cell types (lymphocytes, hepatocytes, and neuronal cells) as examples, the inventors have demonstrated that HIV protease inhibitors were able to modify apoptosis sufficiently in vivo to alter the clinical outcome of apoptotic disease processes. The inventors further observed with in vitro assays that all HIV protease inhibitors tested by the inventors possessed antiapoptotic properties. Without intending to be limited by theory on how HIV protease inhibitors can prevent and treat apoptosis-related diseases and conditions, the inventors demonstrated that the HIV protease inhibitors inhibited the apoptotic signaling at the mitochondria stage. In particular, the HIV protease inhibitors inhibited the mitochondrial adenine nucleotide translocator (ANT) dependent permeability transition pore complex (PTPC) opening. The inventors further demonstrated that although HIV protease inhibitors permitted the premitochondrial signaling events of apoptosis initiated by death receptor stimulation or non-death-receptor signals (e.g., chemotherapeutics), the inhibition at the mitochondria level was sufficient to inhibit apoptosis.

In addition, the present invention can be used to reduce apoptosis in yeast, which is of value to the field of genetically engineered protein synthesis.

In one aspect, the present invention relates to a method of reducing or inhibiting apoptosis in a cell or tissue of a mammal that is not infected with HIV. The method involves administering to said mammal a therapeutically effective amount of one or more HIV protease inhibitors. For example, the method of the present invention can be used to reduce or inhibit the apoptosis of hepatocytes, lymphocytes, neuronal cells, intestinal epithelial cells and cardiomyocytes.

In another aspect, the present invention relates to a method of treating or preventing an apoptosis-related disease or condition in a mammal that is not infected with HIV. The method involves administering to said mammal a therapeutically effective amount of one or more HIV protease inhibitors. For example, the method of the present invention can be used to prevent or treat sepsis and septic shock, stroke, hepatitis, multiple sclerosis, amyotrophic lateral sclerosis, and anthrax toxin or enterotoxin mediated diseases or conditions. Additional examples of the diseases and conditions that can be prevented or treated by the method of the present invention include but are not limited to neural and cardiac ischemia, rheumatoid arthritis, Streptococcal and meningococcal meningitis, neurodegeneration of Alzheimer's disease, Huntington's disease, and viral infections that cause apoptosis (e.g., Rabies and Ebola). When the method is used for prevention purposes, an individual who is at risk of developing an apoptosis-related disease or condition needs to be identified. There are many situations in which such an individual can be identified. For example, during a coronary artery bypass surgery, myocardial ischemia and brain ischemia frequently develop leading to the apoptosis of cardiomyocytes and brain neuronal cells. HIV protease inhibitors can be given to patients who will undergo this surgery to prevent the loss of cardiomyocytes and brain neuronal cells. Brain trauma can also cause brain neuronal cell apoptosis. Thus brain trauma patients can be given HIV protease inhibitors to prevent such cell death.

In another aspect, the present invention relates to a method of reducing or inhibiting apoptosis in a cell or tissue of a mammal that is not infected with HIV. The method involves reducing or inhibiting mitochondrial cytochrome c release in the cell or tissue.

In another aspect, the present invention relates to a method of preventing or treating an apoptosis-related disease or condition in a mammal that is not infected with HIV. The method involves reducing or inhibiting mitochondrial cytochrome c release in target cells or tissues.

In another aspect, the present invention relates to a pharmaceutical kit that contains an HIV protease inhibitor and instruction on how to use the inhibitor to prevent or treat an apoptosis-related disease or condition.

### BRIEF DESCRIPTION OF THE SEVERAL VIEWS OF THE DRAWINGS

FIG. 1 shows the effect of PI treatment on survival in CLP induced sepsis. Mice undergoing CLP were treated with either PI or control for 24 hours prior to cecal perforation, or PI therapy beginning 4 hours after CLP. Survival was monitored for 48 hours.

FIG. 2 shows the impact of PI therapy on aerobic blood cultures. Blood was harvested 12 hours following CLP from vehicle control and PI pretreated animals, and cultured under aerobic conditions.

FIG. 3 shows the impact of PI therapy on plasma TNFα (3A and C), IL-6 and IL-10 (3B and D) levels 6 and 12 hours following CLP in vehicle control and PI pretreated animals.

FIG. 4 shows the impact of PI therapy on 48 hour survival in Rag1−/− mice receiving either vehicle control or PI treatment prior to CLP.

FIG. 5 shows the effects of PI on Jo-2-induced liver failure or SEB induced shock. (A) Mice treated with varying doses of Jo-2 antibody in the presence or absence of PI were followed for 30 days and analyzed for survival. (B) In parallel, mice treated in a similar manner were sacrificed at 4 or 24 hours and analyzed for AST level. (C) Mice treated with SEB plus D-Gal in the presence or absence of PI were analyzed for 24 hour survival, or (D) V-β8 T cell apoptosis by TUNEL.

FIG. 6 shows the effects of PI on two vessel carotid occlusion induced neurodegeneration. Mice treated by two vessel carotid occlusion were treated with or without PI starting 24 hours before, 1 hour after, or 6 hours after carotid occlusion, and analyzed at 48 hours for neurodegeneration in the CA1 region (left panel), denate gyrus (middle panel), or CA3c region (right panel).

FIG. 7 shows the effect of PI on the Fas signaling pathway in vitro and in vivo. (A) Jurkat T cells were stimulated with agonistic anti-Fas in the presence or absence of varying concentrations of PI, and analyzed for Annexin positivity. (B) Caspase 8 and caspase 3 activity, or (C) loss of DiOC₆ retention. Hepatocytes isolated from mice receiving Jo-2 with or without PI (as in FIG. 5) were also analyzed for (D) caspase 8 and caspase 3 activity, or (E) loss of DiOC₆ retention.

FIG. 8 shows the effects of PI on apoptosis in yeast. (A) WT yeast or yeast deficient in both isoforms of VDAC (VDAC Δ1 Δ2) or three isoforms of ANT (ANT Δ1 Δ2 Δ3) were treated with the apoptosis inducing agents Vpr peptide (residues 52-96) or H₂O₂, and analyzed for viability. (B) WT yeast treated with varying doses of PI were treated with the same apoptosis inducers and analyzed for viability.

FIG. 9 shows the effects of PI on the apoptosis inducing abilities of the selective PTPC agonists. (A) Jurkat T cells were treated with either the ANT agonist ATR, VDAC agonist STS, or PBR agonist PK11195, in the presence or absence of PI and analyzed for Annexin positivity, (B) loss of DiOC₆ retention, and (C) caspase 8 and caspase 3 activity.

FIG. 10 shows the effects of PI on PTPC or ANT pore function. (A) Proteoliposomes containing PTPC complexes were treated with PI, stimulated with ATR and analyzed for fluorescence release. (B) proteoliposomes containing ANT were treated with PI, and analyzed for fluorescence release following stimulation with ATR or (C) Vpr peptide (52-96).

### DETAILED DESCRIPTION OF THE INVENTION

The present invention provides methods of reducing or inhibiting apoptosis in a cell or tissue of a mammal not infected with HIV by administering to said mammal a therapeutically effective amount of an HIV protease inhibitor (PI) or a combination of said inhibitors. The present invention further provides that apoptosis in a cell or tissue of a mammal can be reduced or inhibited by reducing or inhibiting mitochondrial adenine nucleotide translocator (ANT) dependent permeability transition pore complex (PTPC) opening in the cell or tissue. The methods provided here can be used to prevent or treat apoptosis-related diseases or conditions in a mammal.

In addition, the present invention can be used to reduce apoptosis in yeast, which is of value to the field of genetically engineered protein synthesis.

Various definitions are provided herein and words specifically defined herein have the meaning provided in the context of the present invention as a whole. Those words not defined herein either explicitly or by context have the broadest meaning consistent with the invention and as understood by those skilled in the art.

As utilized in accordance with the present disclosure, the following terms unless otherwise indicated, shall be understood to have the following meanings.

The terms “effective amount” and “therapeutically effective amount” when used in relation to PI refers to an amount of PI that is useful or necessary to support an observable change in the level of one or more apoptotic processes.

The term “apoptosis,” as is known in the art, describes a type of cellular death that plays an important role during normal development, differentiation, homeostasis or turnover of tissues, and pathological processes as well. Also called “programmed cell death,” this form of cellular demise involves the activation in cells of a built-in genetic program for cell suicide by which cells essentially “autodigest.”

As used herein, “HIV protease inhibitor” is intended to refer to compositions which inhibit HIV protease. Examples include, but are not limited, saquinavir (Roche, Ro31-8959), ritonavir (RIT) (Abbott, ABT-538), lopinavir (Abbott), indinavir (Merck, MK-639), VX-478 (Vertex/Glaxo Wellcome), nelfinavir (NFV) (Agouron, AG-1343), KNI-272 (Japan Energy), CGP-61755 (Ciba-Geigy), U-103017 (Pharmacia and Upjohn), cyclic protease inhibitors, and analogs, mimetics and derivatives thereof. Additional examples of PI may also be used and will be known to one of skill in the art.

In one aspect, the present invention provides methods useful for preventing or treating a wide variety of diseases and pathological conditions where inappropriate apoptosis is involved or has a causal role in the pathophysiology, and is characterized by aberrant levels of apoptotic activity in a cell or tissue. These diseases or conditions in which enhanced apoptosis contributes to the underlying pathogenesis and which are referred to as apoptosis-related diseases and conditions in the specification and claims, include, but are not limited to, neurodegenerative diseases, autoimmune and inflammatory disorders, infectious diseases (including viral hepatitis), inflammatory bowel disease, ischemia/hypoperfusion, and sepsis amongst others.

Neurodegenerative diseases may consist of, but are not limited to, Parkinson's Disease, including early onset forms (Autosomal recessive juvenile Parkinson's; ARJP), Lewy body dementias, and general synucleinopathies; Alzheimer's disease, including frontotemporal dementias (FTD), corticobasal degeneration (CBD), progressive supranuclear palsy (PSP), and general tauopathies and amyloidopathies; Amyotrophic Lateral Sclerosis, including adult-onset motor neuron disease; and Huntington's disease, including spino-cerebellar ataxias and adult onset trinucleotide repeat disorders.

Autoimmune and inflammatory disorders may include, but are not limited to, arthritic diseases such as rheumatoid arthritis, osteoarthritis, gouty arthritis, spondylitis; Behcet disease; sepsis, septic shock, endotoxic shock, gram negative sepsis, gram positive sepsis, and toxic shock syndrome; multiple organ injury syndrome secondary to septicemia, trauma, or hemorrhage; ophthalmic disorders such as allergic conjunctivitis, vernal conjunctivitis, uveitis, and thyroid-associated ophthalmopathy; eosinophilic granuloma; pulmonary or respiratory disorders such as asthma, chronic bronchitis, allergic rhinitis, ARDS, chronic pulmonary inflammatory disease (e.g., chronic obstructive pulmonary disease), silicosis, pulmonary sarcoidosis, pleurisy, alveolitis, vasculitis, pneumonia, bronchiectasis, and pulmonary oxygen toxicity; reperfusion injury of the myocardium, brain, or extremities; fibrosis such as cystic fibrosis; keloid formation or scar tissue formation; atherosclerosis; autoimmune diseases such as systemic lupus erythematosus (SLE), autoimmune thyroiditis, multiple sclerosis, some forms of diabetes, and Reynaud's syndrome; connective tissue disease, autoimmune pulmonary inflammation, Guillain Barre syndrome, autoimmune thyroiditis, insulin dependent diabetes mellitis, myasthenia gravis, graft versus host disease and autoimmune inflammatory eye disease; transplant rejection disorders such as GVHD and allograft rejection, chronic glomerulonephritis; inflammatory bowel diseases such as Crohn's disease, ulcerative colitis and necrotizing enterocolitis, inflammatory dermatoses such as contact dermatitis, atopic dermatitis, psoriasis, or urticaria, fever and myalgias due to infection; central or peripheral nervous system inflammatory disorders such as meningitis, encephalitis, and brain or spinal cord injury due to minor trauma; Sjorgren's syndrome; diseases involving leukocyte diapedesis; alcoholic hepatitis; bacterial pneumonia; antigen-antibody complex mediated diseases; hypovolemic shock; Type I diabetes mellitus; acute and delayed hypersensitivity; disease states due to leukocyte dyscrasia and metastasis; thermal injury; granulocyte transfusion associated syndromes; cytokine-induced toxicity; and allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiform, Stevens Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems.

Infectious diseases amenable to prevention or treatment according to the invention include, but are not limited to, anthrax, bovine spongiform encephalopathy (BSE), chicken pox, cholera, cold, conjunctivitis, Creutzfeldt Jakob Disease (CJD), Dengue fever, diphtheria, ebola, viral encephalitis, Fifth's disease, hand, foot, and mouth disease (HFMD), Hantavirus, Helicobacter Pylori, hepatitis, herpes, hookworm, influenza, Lassa fever, Lyme disease, Marburg hemorrhagic fever, measles, meningitis, mononucleosis, mucormycosis, mumps, nosocomial infections, otitis media, pelvic inflammatory disease (PID), plague, pneumonia, polio, prion diseases, rabies, rheumatic fever, Rocky Mountain spotted fever, roseola, Ross River virus infection, rubella, scarlet fever, sexually transmitted diseases (STDs), shingles, smallpox, Strep throat, tetanus, toxic shock syndrome (TSS), toxoplasmosis, trachoma, tuberculosis, tularemia, typhoid fever, whooping cough, and yellow fever.

Hepatitis, an inflammation of the liver cause by a range of factors including toxins, ischemia, drugs and one of several hepatitis viruses, is another disease amenable to prevention or treatment according to the invention. There are several types of hepatitis virus infections, including hepatitis A, B, and C. Hepatitis A is considered the least threatening since it generally does not lead to liver damage, and 99% of those infected fully recover. Hepatitis B is a serious viral disease that attacks the liver. Approximately 2-10% of adults and 25-80% of children under the age of 5 will not be able to clear the virus in six months and are considered to be chronically infected. Hepatitis C also causes inflammation of the liver, with an estimated 80% of those infected developing chronic hepatitis. Many can develop cirrhosis (scarring of the liver), and some may also develop liver cancer. All forms of hepatitis (both viral and nonviral causes) are amenable to prevention or treatment with the present invention.

Ischemic conditions amenable to prevention or treatment according to the invention include, but are not limited to, cardiac, neural, mesenchymal, and limb ischemia. Ischemia, as set out above, is a condition resulting from insufficient supply of blood, usually caused by arterial blockage, to a tissue or organ. Myocardial infarction and stroke are some of the ischemic conditions amenable to prevention or treatment of the invention.

“Sepsis” is the term used to describe systemic inflammation caused by an overwhelming bacterial infection, pancreatitis, ischemia, toxins etc. Sepsis is a diffuse inflammatory state induced by a variety of potential stimuli including bacterial infection and can also be referred to as systemic inflammatory response syndrome (SIRS). The inflammatory state which ensues is associated with lymphopenia, impaired tissue perfusion, and ultimately end organ dysfunction including brain, lungs, heart, liver, kidney, and muscle. Uncomplicated sepsis, such as that caused by flu and other viral infections, gastroenteritis, or dental abscesses, is very common. Severe sepsis, arises when sepsis occurs in combination with problems in one or more of the vital organs, such as the heart, kidneys, lungs, or liver. Septic shock occurs when sepsis is complicated by low blood pressure that does not respond to standard treatment (fluid administration) and leads to problems in one or more of the vital organs as set out above. This condition has a high mortality rate (around 50%) and is characterized by a lack of oxygen to vital cells, tissues, and organs and extreme reduction in blood pressure.

The diseases and conditions preventable or treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Pharmaceutical Compositions

Pharmaceutical compositions useful in the methods of the invention possess one or more desirable but unexpected combinations of properties, including the ability to decrease apoptosis in a cell or tissue in a mammal or in yeast cells. These compositions are therefore useful for extended prevention or treatment protocols.

The pharmaceutical compositions may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetra-acetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. See Remington's Pharmaceutical Sciences, 18th Edition, A. R. Gennaro, Ed., Mack Publishing Company, 1990.

The optimal pharmaceutical composition will be determined by one skilled in the art depending upon, for example, the intended route of administration, delivery format, and desired dosage. See, for example, Remington's Pharmaceutical Sciences, supra. Such compositions may influence the physical state, stability, rate Of in vivo release, and rate Of in vivo clearance of the specific binding agent.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, binding agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The pharmaceutical compositions can be selected for parenteral delivery (e.g., subcutaneous injection, intramuscular injection, intraperitoneal delivery and intravenous injection). Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this invention may be in the form of a pyrogen-free, parenterally acceptable aqueous solution comprising the desired specific binding agent in a pharmaceutically acceptable vehicle. A particularly suitable vehicle for parenteral injection is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

In another embodiment, a pharmaceutical composition may be formulated for inhalation. For example, a binding agent may be formulated as a dry powder for inhalation. Polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, binding agent molecules that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Another pharmaceutical composition may involve an effective quantity of binding agent in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/00829 that describes controlled release of porous polymeric microparticles for the delivery of pharmaceutical compositions. Additional examples of sustained-release preparations include semipermeable polymer matrices in the form of shaped articles, e.g., films or microcapsules. Sustained release matrices may include polyesters, hydrogels, polylactides (U.S. Pat. No. 3,773,919, EP 58,481), copolymers of L-glutamic acid and gamma ethyl-L-glutamate [Sidman et al., Biopolymers, 22:547-556 (1983)], poly (2-hydroxyethyl-methacrylate) [Langer et al., J. Biomed. Mater. Res., 15:167-277, (1981)] and [Langer et al., Chem. Tech., 12:98-105(1982)], ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc. Natl. Acad. Sci. (USA), 82:3688-3692 (1985); EP 36,676; EP 88,046; EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

Combination Therapy

Pharmaceutical compositions useful in the method of the invention may be used in combination with other therapeutics in the treatment of apoptotic pathologies. The compositions comprising PI may be given in combination with other PI, but they may also contain cytokines, lymphokines, growth factors, or other hematopoietic factors such as members of the TNF superfamily including, for example, TNF-α, TNF-β, TNF0, TNF1, TNF2, interleukins 1-18, members of the interferon (IFN) superfamily, G-CSF, M-CSF, Meg-CSF, GM-CSF, thrombopoietin, stem cell factor, and erythropoietin. Compositions may also include angiopoietins, for example Ang 1, Ang 2, Ang 4, Ang Y, and/or the human angiopoietin like polypeptide, and/or vascular endothelial growth factor (VEGF). Preferred growth factors for use in pharmaceutical compositions of the invention include angiogenin, bone morphogenic proteins 1-15, bone morphogenic protein receptor IA, bone morphogenic protein receptor IB, brain derived neurotrophic factor, ciliary neutrophic factor, ciliary neutrophic factor receptor α, cytokine induced neutrophil chemotactic factor 1, cytokine induced neutrophil, chemotactic factor 2α, cytokine induced neutrophil chemotactic factor 2β, β endothelial cell growth factor, endothelin 1, epidermal growth factor, epithelial derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line derived neutrophic factor receptor α1, glial cell line derived neutrophic factor receptor α2, growth related protein, growth related protein α, growth related protein β, growth related protein γ, heparin binding epidermal growth factor, hepatocyte growth factor, hepatocyte growth factor receptor, insulin like growth factor I, insulin like growth factor receptor, insulin like growth factor II, insulin like growth factor binding protein, keratinocyte growth factor, leukemia inhibitory factor, leukemia inhibitory factor receptor α, nerve growth factor nerve growth factor receptor, neurotrophin 3, neurotrophin 4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase type plasminogen activator receptor, vascular endothelial growth factor, and chimeric proteins and biologically or immunologically active fragments thereof. Compositions may also include chemokines such as MIP-1α, MIP-1β, RANTES, SDF-1, BOB, and BONZO etc.

The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. A protein of the present invention may be active in multimers (e.g., heterodimers or homodimers) or complexes with itself or other proteins. As a result, pharmaceutical compositions of the invention may comprise a protein of the invention in such multimeric or complexed form.

HIV PI used in the present invention may be administered in accordance with the method of the invention either alone or in combination with other therapies such as treatments employing cytokines, lymphokines or other hematopoietic factors. When co-administered with one or more cytokines, lymphokines or other hematopoietic factors, PIs of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Dosing and Formulation

The HIV PI treatment of the invention can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chose route of administration and standard pharmaceutical practice.

Pharmaceutical compositions of the invention can be administered at the dosage known in the art for the treatment of inhibition of HIV infection and can be modified depending on, e.g., the severity of the condition, the site of apoptosis, the nature and extent of symptoms, kind of concurrent treatment, frequency of treatment, and the effect desired. The dosages administered to the non-HIV infected patient may be greater depending on the pathological condition of patient, i.e., for example, for sepsis or stroke. The dosage will also vary depending on the health, sex, age, and diet of the recipient, time of administration, as well as other clinical factors. The addition of other PIs or other factors to the final composition, may also affect the dosage.

For use in the treatment of inhibition of the replication of HIV, by way of general guidance, a daily oral dosage of active ingredient can be about 0.001 to 1000 mg/kg of body weight. Ordinarily a dose of 0.1 to 500 mg/kg per day in divided doses one to four times a day or in sustained release form is effective to obtain the desired results. Dosage forms (compositions) suitable for administration contain about 1 milligram to 100 milligrams of active ingredient per unit. In these pharmaceutical compositions the active ingredient will ordinarily be present in an amount of about 0.5-95% by weight based on the total weight of the composition.

For any compound, the therapeutically effective dose can be estimated initially either in cell culture assays or in animal models such as mice, rats, rabbits, dogs, or pigs. An animal model may also be used to determine the appropriate concentration range and route of administration. Such information can then be used to determine useful doses and routes for administration in humans.

The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration. For example, the active ingredients may be administered by a subcutaneously implanted capsule which releases active ingredients for up to several months.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient. For example, tissues harvested for use in transplantation may be treated ex vivo according to the present invention and subsequently transplanted into a recipient. The treatment would prolong the duration that the graft can be stored prior to implantation.

In other cases, an agent can be delivered by implanting certain cells that have been genetically engineered, using methods such as those described herein, to express and secrete the agent. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

The active ingredient can be administered orally in solid dosage forms, such as capsules, tablets, and powders, or in liquid dosage forms, such as elixirs, syrups, and suspensions. It can also be administered parenterally, in sterile liquid dosage forms.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

Suitable pharmaceutical carriers and methods of preparing pharmaceutical dosage forms are described in Remington's Pharmaceutical Sciences, Mack Publishing Company, a standard reference text in this field.

The combination products of this invention may be formulated such that, although the active ingredients are combined in a single dosage unit, the physical contact between the active ingredients is minimized. In order to minimize contact, for example, where the product is orally administered, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time or concurrently by the same manner, will be readily apparent to those skilled in the art, based on the present disclosure.

Pharmaceutical Kits

Pharmaceutical kits useful for the prevention or treatment of apoptosis, which comprise a therapeutically effective amount of a pharmaceutical composition comprising a compound and optionally one or more components, in one or more sterile containers, are also within the ambit of the present invention. Sterilization of the container may be carried out using conventional sterilization methodology well known to those skilled in the art. The component(s) may be in the same sterile container or in separate sterile containers. The sterile containers of materials may comprise separate containers, or one or more multi-part containers, as desired. Component(s), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

### EXAMPLES

The present invention is described in more detail with reference to the following non-limiting examples, which are offered to more fully illustrate the invention, but are not to be construed as limiting the scope thereof. The examples illustrate that HIV protease inhibitors also block apoptosis induced by a variety of non-viral stimuli in vivo with consequent reduction in mortality. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

### Mouse Pharmacokinetic Studies with Protease Inhibitors

To determine optimum dosing and physiological levels of protease inhibitors obtained in vivo in mice, pharmacokinetic studies along with the measurement of physiological levels of PI in the plasma and brains of mice, and histopathological studies were carried out.

Administration of PI for Experimental Protocols: Six to eight-week-old BALB/C mice (Jackson Labs, Bar Harbor, Me.) were acclimatized for one week before receiving either control vehicle or PI by gavage. PIs were prepared by dissolving 1% weight per volume carboxymethylcellulose and 15 mg/mL Nelfinavir (NFV) oral powder (commercial preparation, Agouron Laboratories, La Jolla, Calif.) in 2% ethanol in distilled water. Where indicated, 3 μL Ritonavir (RIT) (commercial preparation, liquid suspension, Abbott Laboratories, North Chicago, Ill.) was added to 200 μL of NFV to yield final drug concentrations of 125 mg/kg NFV and 13 mg/kg RIT. The PI mixture was administered every 8 hours for the indicated number of doses and mice were either sacrificed for pharmacokinetic studies or treated as described. Histopathology was performed by a consulting veterinary pathologist who was blinded as to the nature of experimental treatments.

Pharmacokinetic Studies: Six to eight week old mice (BALB/C) received a slurry of NFV every 8 hours by gavage at doses of either 125 mg/kg or 250 mg/kg. Immediately preceding the fourth dose and every two hours post gavage until hour 8, plasma drug concentrations were assayed. At both doses, NFV was undetectable (i.e. <2.5 ng/mL) 6 hours after dosing.

Because RIT is a potent inhibitor of intestinal and hepatic CY3A4 isoenzyme P450 activity [Kempf, In: Protease Inhibitors in AIDS Therapy (ed. Flexner, C. W.) 49-64 (Mark Dekker, New York, 2001)], the co-administration of RIT with NFV in human subjects significantly increased levels of NFV compared to NFV alone (Raines et al., J. Acq. Immune Defic. Synd. 25:322-8, 2000). Another group of mice were therefore treated with NFV 125 mg/kg and RIT 13 mg/kg to exploit the inhibitory effect of RIT on the metabolism of NFV.

Drug levels were assessed in two mice each for several time-points. NFV levels were 8172 and 8914 ng/mL at 1 hour, 5999 and 6006 ng/mL at 2 hours, 5559 and 6048 ng/mL at 3 hours, and 4005 to 4217 ng/mL at 4 hours post-dosing. NFV trough concentrations (8 hours post-dosing) ranged from 1199 to 1258 ng/mL, a level comparable to trough concentrations observed in HIV-1 infected adults using NFV in the approved dose of 1250 mg twice daily (Baede-van Dijk et al., Aids 15:991-8, 2001). Similar drug concentrations were seen in the plasma of ND4 and C57BL/6 mice that received the same doses. This dosing regimen (NFV 125 mg/kg and RIT 13 mg/kg) was chosen to assess the antiapoptotic effects of the combination NFV and RIT.

Analysis of NFV and RIT Levels in Mouse Plasma/Tissues: Plasma concentrations of NFV and RIT were measured simultaneously using liquid chromatography with tandem mass-spectrometry (LC/MS/MS). The lower limit of quantitation for NFV and RIT was 2.5 ng/mL. The accuracy of quality control (QC) samples, analyzed simultaneously with the study samples, ranged from 83.7 to 107.5% for NFV, and 105.0 to 116.5% for RIT. The within-run variability for the measurement of three replicate analyses of the QC samples was less than 9.7% for both NFV and RIT. This assay has been cross-validated as part of the International Interlaboratory Quality Control Program for Therapeutic Drug Monitoring in HIV Infection (Aarnoutse et al., Antimicrob. Agents Chemother. 46:884-886, 2002). A recent survey showed that the accuracy for the measurement of NFV and RIT over three different concentrations ranged from 88.1 to 93.6%, and 97.0 to 106.3%, respectively.

To determine if mice treated with NFV/RIT suspension achieved detectable brain drug levels of PI, the following experiment was carried out. Two mice received 3 doses of NFV/RIT suspension, and immediately preceding the fourth dose, were euthanized and the whole brain was removed. NFV and RIT concentrations in brain tissues were determined after the tissues were washed extensively with PBS, blotted dry with filter paper, weighed, and placed in 5 ml of 0.1 M phosphoric acid and homogenized using a polytron. Supernatants were then analyzed as described herein for drug level determination. Levels of NFV were 682 μg/gm of brain tissue and 986 μg/gm of brain tissue, respectively. RIT levels were 38 μg/gm and 32 μg/gm of brain tissue, respectively. Given the significant drug levels achieved in the brains of these mice, the effects of PI in a mouse model of stroke could be examined.

The data as set out above demonstrated that the described treatments with PI resulted in sufficient physiological levels of PI in the sera and brains of mice to carry out in vivo studies as described herein.

### Example 2

### Improved Survival in Experimental Sepsis with Oral Protease Inhibitors Administered as Either a Prevention or a Therapeutic Agent

Sepsis is characterized first by a hyperimmune, hyperinflammatory state with a Th1 predominant cytokine profile (e.g. IFNγ, IL-2, and TNFα), followed by a hypoimmune, hypoinflammatory state, characterized by a Th2 cytokine profile (e.g. IL-4, 6, 10). This model is supported by observations in septic patients including loss of delayed type hypersensitivity (Meakins, J. L. et al. Ann Surg 186, 241-250, 1977), an inability to clear infection (Lederer, J. A. et al. Shock 11, 153-159, 1999), and a predisposition to nosocomial infections (Oberholzer, A. et al. Shock 16, 83-96, 2001). Potential mechanisms of immune suppression in septic patients include: a shift from a Th1 to a Th2 response (Gogos, C. A. et al. J Infect Dis 181, 176-180, 2000), anergy (Heidecke, C. D. et al. Am J Surg 178, 288-292, 1999; Pellegrini, J. D. et al. J Surg Res 88, 200-206, 2000), loss of macrophage expression of major histocompatability complex (MHC) class II and co-stimulatory molecules (Ayala, A. et al. Shock 5, 79-90, 1996), immunosuppressive effects of apoptosis (Green, D. R., and Beere, H. M. Nature 405, 28-29, 2000; Voll, R. E. et al. Nature 390, 350-351, 1997; Fadok, V. A. et al. Nature 405, 85-90, 2000), and apoptosis of CD4 T cells, B cells, and dendritic cells, leading to decreased antibody production, macrophage activation, and antigen presentation, respectively (Hotchkiss, R. S. et al. Crit Care Med 27, 1230-1251, 1999; Hotchkiss, R. S. et al. J Immunol 166, 6952-6963, 2001; Hotchkiss, R. S. et al. J Immunol 168, 2493-2500, 2002). In this example, we show that reduced T cell apoptosis led to improved outcomes, improved control of bacterial replication, and enhanced proinflammatory cytokine production early in the septic response. Due to the pervasive role of CD4 T cells in orchestrating a coordinated adaptive immune response, we predict that inhibition of lymphocyte apoptosis can improve other immune parameters as well.

For example, it has recently been described that how a cell dies can impact the immunologic function of surviving immune cells (Hotchkiss, R. S. et al. Crit Care Med 27, 1230-1251, 1999; Hotchkiss, R. S. et al. J Immunol 166, 6952-6963, 2001; Hotchkiss, R. S. et al. J Immunol 168, 2493-2500, 2002). Uptake of apoptotic cellular debris by phagocytic cells stimulates immune tolerance by the release of antiinflammatory cytokines including IL-6 and IL-10 (Hotchkiss, R. S. et al. Crit Care Med 27, 1230-1251, 1999; Hotchkiss, R. S. et al. J Immunol 166, 6952-6963, 2001; Fadok, V. A. et al. J Clin Invest 101, 890-898, 1998; McDonald, P. P. et al. J Immunol 163, 6164-6172, 1999; Chen, W. et al. Immunity 14, 715-725, 2001). In addition, uptake of apoptotic cells by macrophages and dendritic cells impairs expression of costimulatory molecules (Barker, R. N. et al. Pathobiology 67, 302-305, 1999; Sauter, B. et al. J Exp Med 191, 423-434, 2000). Thus, T cells that come into contact with antigen presenting cells that have ingested apoptotic cells are inadequately stimulated and became anergic hyporesponsive, or undergo apoptosis themselves (Green, D. R., and Beere, H. M. Nature 405, 28-29, 2000). Such apoptosis induced immune suppression is highlighted by the adoptive transfer of apoptotic splenocytes into septic mice, which worsens survival compared to transfer of necrotic splenocytes (Hotchkiss, R. S. et al. Proc Natl Acad Sci USA 100, 6724-6729, 2003). In our system, reduced T cell apoptosis associated with PI administration was associated with reduced IL-6 and IL-10 production late in the septic response. One potential mechanism by which this may have occurred is through reduced phagocytic uptake of apoptotic bodies resulting in Th2 immune skewing and therefore less immune suppression.

An additional potential benefit of apoptosis inhibition in sepsis may be related to the enhanced bowel epithelial apoptosis which is seen in both animal models and studies of humans with prolonged sepsis (Coutinho, H. B. et al. J Clin Pathol 50, 294-298, 1997; Hiramatsu, M. et al. Shock 7, 247-253, 1997; Sileri, P. et al. Dig Dis Sci 47, 929-934, 2002; Cinel, I. et al. Pharmacol Res 46, 119-127, 2002). The impact of gut epithelial apoptosis in sepsis is unknown, but it may reduce the barrier function of the gut and promote translocation of intestinal bacteria which in turn may exacerbate the septic response. Inhibition of sepsis induced epithelial apoptosis would predictably reduce the bacterial burden. Finally, sepsis induced tissue hypoperfusion is common in sepsis and results clinically in impaired end organ function including oligemia and acute tubular necrosis (Sayeed, M. M. Crit Care Med 31, 1864-1866, 2003), mental status changes due to neuronal ischemia (Wilson, J. X., and Young, G. B. Can J Neurol Sci 30, 98-105, 2003; Gray, H. W. Semin Nucl Med 32, 159-172, 2002), reduced cardiac output (Fowler, D. E., and Wang, P. Int J Mol Med 9, 443-449, 2002), etc. Emerging data suggests that on the cellular level, tissue hypoperfusion results in anoxic apoptosis (Hotchkiss, R. S. et al. Crit Care Med 25, 1298-1307, 1997; Power, C. et al. Shock 18, 197-211, 2002), for example, of renal tubular epithelial cells (Ortiz, A. et al. Biochem Pharmacol 66, 1589-1594, 2003). Therefore, apoptosis inhibition may improve these hypoperfusion related complications of sepsis as well.

Much of the cumulative data regarding sepsis can be reconciled in a model whereby the initial sepsis event (e.g., bacteremia) promotes lymphocyte activation and production of Th1 cytokines (Volk, H. D. et al. Intensive Care Med 22 Suppl 4, S474-481, 1996) including IL-2 and IFNγ which are beneficial to the host (Goronzy, J. et al. J Immunol 142, 1134-1138, 1989; Docke, W. D. et al. Nat Med 3, 678-681, 1997). Subsequently, enhanced lymphocyte activation and proliferation leads to enhanced lymphocyte apoptosis, immunosuppression, anergy loss of Th1 cytokine production and a shift towards a Th2 profile. Indeed, in septic patients, each of these events are seen; lymphocytosis followed by lymphopenia, a shift in plasma cytokines from Th1 to Th2 predominance (Gogos, C. A. et al. J Infect Dis 181, 176-180, 2000), loss of delayed hypersensitivity responses late in sepsis (Meakins, J. L. et al. Ann Surg 186, 241-250, 1977; Lederer, J. A. et al. Shock 11, 153-159, 1999), and high numbers of apoptotic lymphocytes in late sepsis both in animal models (Wang, S. D. et al. J Immunol 152, 5014-5021, 1994; Ayala, A. et al. Blood 87, 4261-4275, 1996; Hotchkiss, R. S. et al. Crit Care Med 25, 1298-1307, 1997; Hotchkiss, R. S. et al. Science 294, 1783, 2001; Chung, C. S. et al. Am J Physiol Gastrointest Liver Physiol 280, G812-818, 2001. Freeman, B. D. et al. Crit Care Med 28, 1701-1708, 2000; Hotchkiss, R. S. et al. J Immunol 164, 3675-3680, 2000; Tinsley, K. W. et al. Shock 13, 1-7, 2000; Ayala, A. et al. Blood 91, 1362-1372, 1998) and human autopsy studies (Hotchkiss, R. S. et al. Crit Care Med 27, 1230-1251, 1999; Coutinho, H. B. et al. J Clin Pathol 50, 294-298, 1997). In this example, we show that preventing apoptosis has functional consequences of decreased blood bacterial counts, increased TNFα early in sepsis, attenuated Th2 cytokine profile in late sepsis and improved survival in experimental murine CLP induced sepsis. These effects are not due to direct antibacterial properties of PI, and the survival benefit is not seen in animals lacking lymphocytes. Thus, lymphocyte apoptosis in sepsis may both directly and indirectly impact host outcome and therefore be an appropriate target for therapeutic intervention.

Materials and Methods

Cecal Ligation and Perforation: Female ND4 (Harlan, Indianapolis, Ind.) and Rag1−/− and their appropriate C57BL/6 controls (Jackson Laboratory, Bar Harbor, Me.), 6-8 weeks, 15-20 g were housed in a germ free environment and given access to standard laboratory chow and water. The murine CLP model that reproduces many of the clinical features of sepsis in patients was used to induce intra-abdominal sepsis (Chaudry, I. H. et al. Surgery 85, 205-211, 1979). This model is a clinically relevant model of sepsis that has been validated in many laboratories (Remick, D. G. et al. Shock 13, 110-116, 2000; Baker, C. C. et al. Surgery 94, 331-335, 1983). Briefly, following externalization of the cecum, 2 perforations were made on the antimesenteric border of the cecum with a 19 gauge needle. Post-operatively, mice were resuscitated with 1 cc of normal saline subcutaneously, and were given access to standard laboratory chow and acetaminophen supplemented water. Animals were euthanized if they displayed any of the following characteristics: moribund, lateral recumbency, and/or hypothermia (rectal temperature <32° C.). All surviving animals were euthanized at 48 hrs.

All animal treatments were reviewed and approved by the Mayo Foundation Institutional Animal Care and Use Committee.

PI Treatments: Beginning at the indicated times, animals were treated every 8 hours by oral gavage with a PI mixture consisting of 125 mg/kg nelfinavir (Agouron Pharmaceuticals, La Jolla, Calif.) and 13 mg/kg ritonavir (Abbott Pharmaceuticals, Abbott Park, Ill.) in 2% ethanol in distilled water, or vehicle control (2% ethanol). Pharmacokinetic studies in mice demonstrated that with this dosing regimen, nelfinavir plasma levels were similar to those achieved in humans receiving a standard dose of nelfinavir (data not shown). In order to achieve these levels, it was necessary to co-dose nelfinavir with a small dose of ritonavir (another PI), which inhibits CYP3A mediated nelfinavir metabolism, thus increasing nelfinavir plasma levels.

Immunohistochemistry: At the time of euthanasia, the heart, lungs, thymus, liver, spleen, kidneys and ileum were harvested and placed in 10% neutral buffered formalin (Sigma, St. Louis, Mo.). A random selection of tissues from 2 animals per treatment group (pretreated and vehicle control) was made. These tissues were embedded in paraffin and terminal deoxynucleotide UTP transferase (TUNEL) staining performed (Molecular Histology, Little Rock, Ark.). Quantification of apoptotic cells was performed by a panel of 3 blinded reviewers. TUNEL positive and negative cells were enumerated, and TUNEL positivity expressed as a percentage of total cells counted. The scores of the 3 reviewers were averaged to derive the final percentage.

Microbiology: To evaluate for any antibacterial effects of PI, we performed standard minimal inhibitory concentration (MIC) analyses of nelfinavir against Pseudomonas aeruginosa, Enterococcus, Eschericheria coli, and Streptococcus bovis. Five different clinical isolates of each organism were tested according to the National Committee for Clinical Laboratory Standards (NCCLS) guidelines (National, and Standards, C. L. (2003) Methods for dilution antimicrobial susceptibility tests for bacteria that grow aerobically; approved standard-sixth edition. In Approved standards 6th edition (M7-A6, N. d., ed), Wayne, Pa.). Organisms were subcultured twice from freezer stocks, then incubated in Tryptic Soy Broth to logarithmic growth. These cultures were diluted to 10⁵ colony forming units (cfu) per ml and tested. Nelfinavir was dissolved in DMSO to 1000 μM, and further diluted in Mueller Hinton Broth.

For quantitative blood cultures, blood was obtained via cardiac puncture from pretreated and vehicle control animals at 12 hours post perforation. 0.1 ml of blood was diluted in 0.9 ml of sterile saline, plated in 10-fold serial dilutions on blood agar plates (Becton Dickinson, Cockeysville, Md.), and aerobically incubated at 37° C. Plates with between 10 and 100 colonies were used for quantification of the cfu per mL of blood.

Plasma Cytokines: Blood was obtained via heparinized ventricular puncture at 6 or 12 hours post-perforation from animals in the pretreated and vehicle control groups. Following centrifugation (14,000 rpm, 5 min, 4° C.), the plasma was removed and stored at −80° C. Using standard ELISA procedures as outlined by the manufacturer (Bio-Rad, Hercules, Calif.), determination of the plasma levels of TNFα, IL-6, and IL-10 were performed. The lower limit of quantification for each cytokine was 6.9 pg/ml.

Statistical Analyses. Survival analyses were performed by Kaplan-Meier analysis. Blood counts, cytokines, and TUNEL positive cells were analyzed with student's t-test.

Results

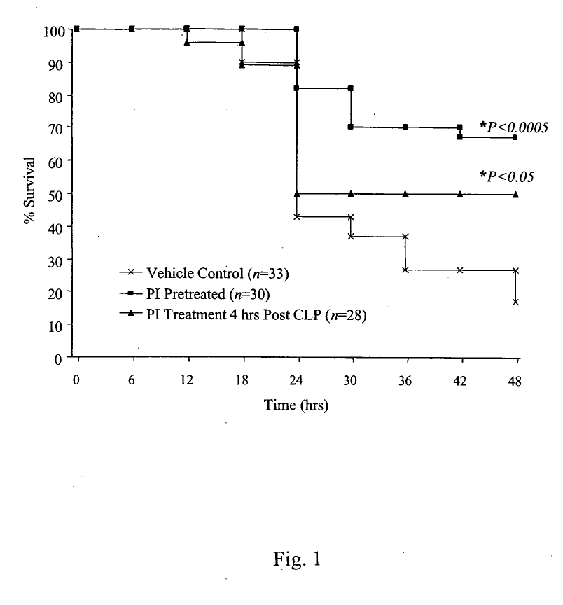

PI Therapy Is Associated With Improved Survival in Sepsis: To evaluate the impact of PI therapy on survival in sepsis, animals were gavaged with PI cocktail (nelfinavir and ritonavir) beginning either 24 hours prior to (n=33), or 4 hours after (n=28) cecal perforation. Control animals (n=30) received vehicle control alone beginning 24 hours prior to perforation. Sham operated animals (n=10) underwent laparotomy and cecal manipulation, but no ligation nor perforation was performed. Survival at 48 hours was 17% in the control group, 67% (P<0.0005) in the pretreated group, and 50% (P<0.05, compared with control) when treatment began 4 hours after perforation (FIG. 1). Sham operated animals experienced no deaths.

PI Therapy Attenuates Lymphocyte Apoptosis in the Spleen and Thymus: To investigate whether the observed survival advantage conferred by PI was associated with a reduction in apoptosis, TUNEL staining of all major organs was performed. No appreciable apoptosis was detected in the heart, lungs, liver, ileum, or kidneys. Control animals demonstrated intense TUNEL staining in the spleen 41%, and thymus 59%. In contrast, those animals pretreated with PI displayed reduced splenic and thymic TUNEL positivity of 8% (P<0.05) and 14% (P<0.05), respectively.

PIs Do Not Possess Antibacterial Activity: One possible explanation for the improved survival observed with PI treatment is that PI may possess unanticipated antibacterial properties. We therefore used NCCLS Guidelines to formally test whether nelfinavir had intrinsic antibacterial effects against P. aeruginosa, Enterococcus, E. coli, and S. bovis. In all cases, the MIC for nelfinavir was greater than 10 μM. In our pharmacokinetic studies in mice (data not shown), and in humans receiving nelfinavir, Cₘₐₓ is approximately 8 μM (Pellegrin, I. et al. Aids 16, 1331-1340, 2002). Thus, even at concentrations greater than those observed clinically, nelfinavir does not inhibit bacterial growth.

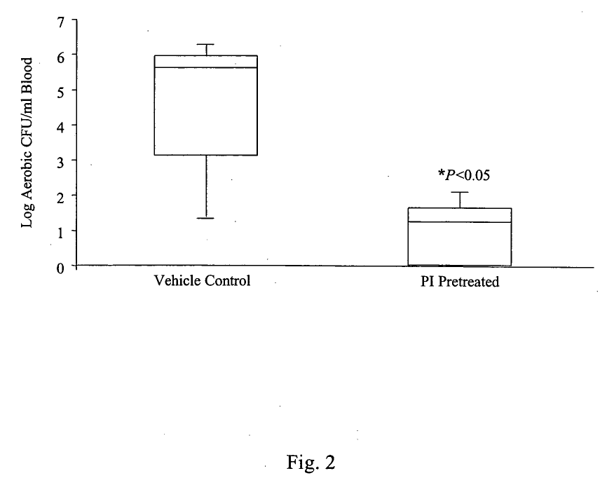

With the knowledge that PI possesses no antibacterial properties in vitro, we next looked at the effect of PI therapy on bacterial blood counts. Twelve hours following perforation, control animals had a mean bacterial blood count of log₁₀ 5.8/mL, while pretreated animals had an average of log₁₀ 1.6/mL (P<0.05) (FIG. 2).

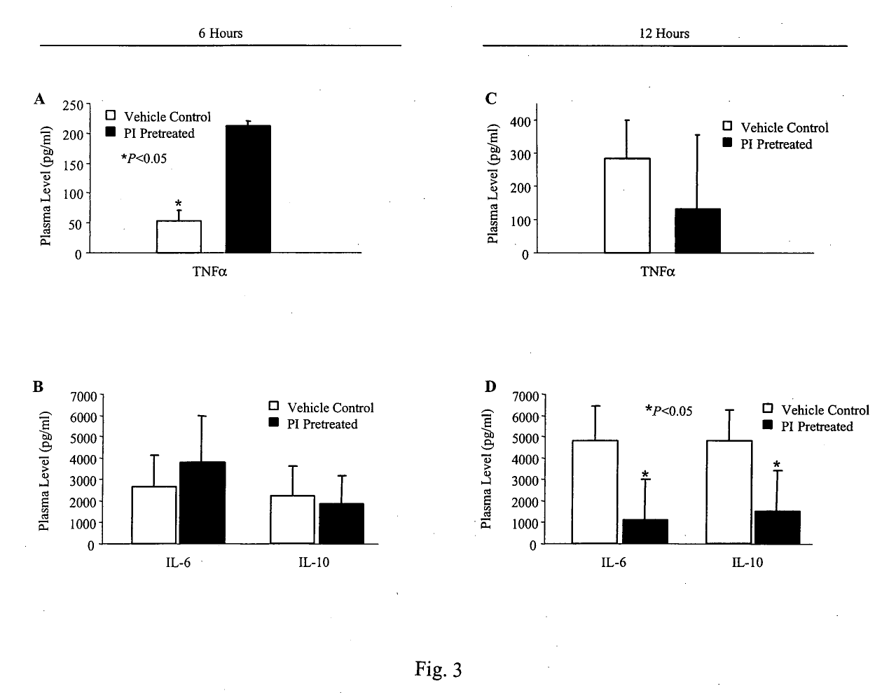

Protease Inhibitor Therapy Affects the Biphasic Plasma Cytokine Profile in Sepsis: Cytokine profiles in sepsis are biphasic, with a proinflammatory/Th1 predominance early in sepsis and an antiinflammatory/Th2 profile late in sepsis. To examine the impact of PI treatment on plasma cytokine profile, we looked at the prototypic Th1 cytokine TNFα, and Th2 cytokines IL-6 and IL-10. Early in sepsis (6 hours), PI treatment was associated with an elevation of TNFα from 54 pg/ml in control treated animals to 212 pg/ml (P<0.05) (FIG. 3A). There was no significant impact on IL-6 or Il-10 levels at 6 hours (FIG. 3B). However, by 12 hours after perforation, both IL-6 and IL-10 plasma levels in PI pretreated animals were significantly lower (1139 pg/ml and 1496 pg/ml, respectively; P<0.05) when compared to IL-6 and IL-10 levels in vehicle control treated animals (4809 pg/ml and 4815 pg/ml, respectively) (FIG. 3D). TNFα plasma levels at 12 hours were not significantly altered (FIG. 3E). Thus, PI augment the proinflammatory response early in sepsis, yet attenuate the Th2 predominant cytokine profile normally seen late in sepsis.

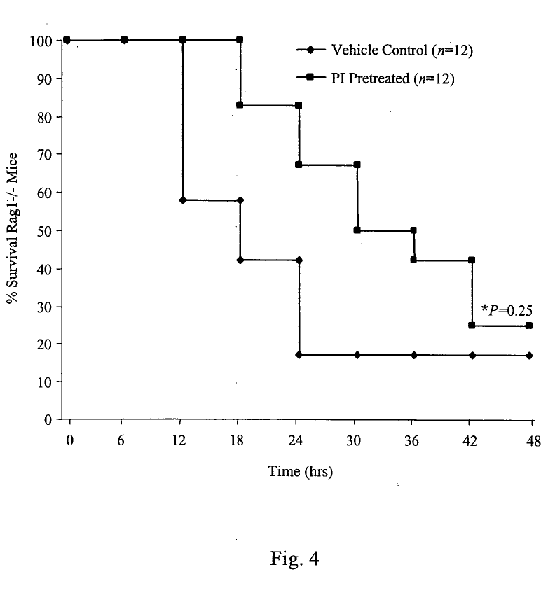

Protease Inhibitor Therapy Does Not Alter Survival in Rag1−/− Mice: To examine the significance of PI mediated inhibition of lymphocyte apoptosis, we repeated these studies using mice lacking functional lymphocytes. Rag1−/− mice on a C57BL/6 background were either pretreated with PI (n=12) or vehicle control (n=12) for 24 hours prior to perforation. Forty-eight hours following CLP, there was no difference in survival between the PI and vehicle control treated Rag1−/− mice (25% vs 17% respectively, P=0.25) (FIG. 4). Repeating these experiments with wild type C57BL/6, survival results similar to those with ND4 mice were obtained.

### Example 3

### Inhibition of Pathologic Apoptosis In Vivo by HIV Protease Inhibitors as a New Prevention and Therapeutic Strategy for Hepatitis, Staphylococcal Enterotoxin B-Induced Shock, Stroke and Yeast Apoptosis

Materials and Methods

Mouse treatments: Mice received either PI in 2% ethanol or vehicle control (2% ethanol) by oral gavage every eight hours. Animal treatments were reviewed and approved by the Mayo Foundation IACUC and University of Ottawa Animal Care Committee. Plasma concentrations of NFV and RIT were measured simultaneously using liquid chromatography with tandem mass-spectrometry. The lower limit of quantitation for NFV and RIT was 2.5 ng/mL. The accuracy of quality control samples analyzed simultaneously with the study samples ranged from 83.7 to 107.5% for NFV, and 105.0 to 116.5% for RIT. The within-run variability for three replicative analyses of the quality control samples was less than 9.7% for both NFV and RIT. This assay has been cross-validated as part of the International Interlaboratory Quality Control Program for Therapeutic Drug Monitoring in HIV Infection (Aarnoutse, R. E. et al. Antimicrob Agents Chemother 46, 884-6, 2002). To determine a PI dosing regimen that would result in relevant plasma concentrations, 6 to 8 week old BALB/C and C57BL/6 mice received 250 mg/kg of NFV (Agouron, La Jolla, Calif.) every 8 hours, yet plasma concentrations were undetectable 6 hours after dosing. Since co-administration of RIT in humans significantly increases NFV levels compared to NFV alone (Raines, C. P. et al. J Acquir Immune Defic Syndr 25, 322-8, 2000), we treated mice every 8 hours with NFV 125 mg/kg and RIT (Abbott Labs, Abbott Park, Ill.) 13 mg/kg. Eight-hour NFV trough levels ranged from 1199 to 1258 ng/mL, which are comparable to concentrations observed in HIV infected patients using NFV. We also assessed whether C57BL/6 mice treated with this dose of NFV/RIT achieved detectable brain drug levels. NFV and RIT concentrations in brain tissues were determined after the tissues were washed extensively with PBS, blotted dry with filter paper, placed in 5 mls of 0.1M phosphoric acid and homogenized using a polytron. Supernatants were analyzed as above for drug level determination. Analysis of whole brains of PI treated mice revealed levels of NFV between 682 μg/gm and 986 μg/gm of brain tissue. We therefore opted to assess the in vivo antiapoptotic effects of the combination of NFV and RIT using this dosing regimen.

Fas Induced Liver Failure: Six-week-old female C57BL/6 mice (Charles River, Wilmington, Mass.) received the indicated doses of Jo-2 anti-Fas antibody (BD Pharmingen, San Diego, Calif.) by tail vein injection. Moribund animals, or animals who survived until the indicated time points were sacrificed using CO₂ asphyxiation. Clinical chemistries were performed on serum obtained from 3 mice from each treatment group at 4 and 24 hours after Jo-2 injection. Quantification of hepatocyte apoptosis by TUNEL (Roche, Indianapolis, Ind.) positivity was performed by four blinded reviewers, and the results averaged.

Staphylococcal Enterotoxin B Induced Shock: Six-week-old female BALB/c mice (Harlan, Indianapolis, Ind.) received 20 mg of D-gal (Sigma, St. Louis, Mo.) and 6.5 μg SEB (Toxin Technology, Sarasota, Fla.) intraperitoneally. Animals were euthanized if they became moribund, laterally recumbent, or hypothermic (rectal temperature <32° C.), or at the indicated time points. As necessary, spleens were harvested, and splenocytes isolated mechanically with plastic mesh. Following red cell lysis and B cell removal by incubation with nylon wool (1 hour, 37° C.; Accurate Chemical and Scientific Corporation, Westbury, N.Y.), purified T cells were stained with monoclonal anti-mouse Vβ8-FITC antibody, or appropriate isotype control (BD Pharmingen), followed by wash with PBS and fixation with 1% paraformaldehyde. Dual staining with TUNEL-TMR Red (Roche) was performed as per manufacturer's instructions.

Two-vessel Cerebral Occlusion: Male C57BL/6 mice (Charles River), weighing 16-18 g, were anaesthetised with isofluorane, and the common carotid arteries exposed. Blood flow was interrupted with a silk suture, and restored after 10 minutes. Neuronal damage was monitored by histologic analysis of animals sacrificed 48 hours following ischemia. Briefly, animals were euthanized with CO₂, decapitated, and brains were rapidly removed, frozen in isopentane and stored at −80° C. Ten micron sections were cut on a cryostat, fixed in 1% glutaraldehyde, and stained with H & E (Bennett, S. A. et al. Neuroreport 9, 161-6, 1998). Cells that were pyknotic, eosinophilic, or hyperchromatic with amorphous or fragmented nuclei were considered damaged. The number of degenerating cells in the CA1, CA3c, and superior limb of the dentate gyrus granule cell layer per section were counted bilaterally at ×200 magnification. Counts were averaged across both hemispheres to yield a single value for each animal.

Cell Culture and Apoptosis Induction In Vitro: Jurkat T cells (ATCC, Rockville, Md.) were cultured in RPMI media (Mediatech, Herndon, Va.), supplemented with 10% heat inactivated fetal bovine serum (Atlanta Biologicals, Norcross, Ga.), and penicillin/streptomycin (Gibco, Carlsbad, Calif.) at 37° C. in 5% CO₂. Where indicated, cells were incubated with 7 μM NFV or vehicle (DMSO) for 6 hours, prior to induction of apoptosis with indicated doses of CH-11 anti-Fas antibody (Upstate, Waltham, Mass.), ATR (Sigma), STS (Calbiochem, San Diego, Calif.), or PK11195 (Sigma).

Hepatocyte isolation: Four hours after treatment with 7.5 μg of Jo-2 antibody, mice were euthanized with pentobarbital (60 mg/kg, Abbott), and hepatocytes isolated via a two-step collagenase digestion (Seglen, P. O. Methods Cell Biol 13, 29-83, 1976). Following red cell lysis, hepatocytes were enumerated and evaluated for loss of ΔΨₘ, and caspase 8 and 3 activity.

Assays of Apoptotic Signaling: TUNEL. Briefly, 1×10⁶ cells were fixed with 2% paraformaldehyde, permeabilized with 0.1% Triton X-100 and 0.1% citrate, and stained with TUNEL as per manufacturer's directions (Roche).

Annexin-V and Propidium Iodide Staining. Annexin binding buffer (BD Pharmingen) was added to 1×10⁶ cells in 500 μl of media followed by 2 μl of Annexin V-FITC (BD Pharmingen) and 1 μl of Propidium Iodide (Sigma). The mixture was incubated for 30 minutes at 37° C., followed by FACS analysis.

Western Blot Analysis. Whole cell lysates were subjected to SDS-PAGE. Following transfer, PVDF membranes (Millipore, Billerica, Mass.) were probed with antibodies to caspase 3 (1 μg/ml; Gene Therapy Systems, San Diego, Calif.), caspase 9 (1 μg/ml MBL, Naka-ku, Nagoya, Japan), cytochrome c (1 μg/ml; BD Parmingen), or PARP (1 μg/ml; BD Pharmingen). Following incubation with goat anti-mouse HRP (1:10000; Amersham Bioscience, Piscataway, N.J.), enhanced chemiluminescence (Amersham Bioscience) was used to detect proteins of interest.

Fluorometric Caspase 8 and 3 Activity. Aliquots of 5×10⁶ lysed cells were resuspended in reaction buffer with the appropriate fluorogenic caspase substrate (IETD for caspase 8 and DEVD for caspase 3) (R&D Systems, Minneapolis, Minn.). Activity was determined with a fluorescence plate reader (BioTek, Winooski, Vt.) at an excitation wavelength of 400 nm and emission of 505 nm. DiOC₆ 1×10⁶ intact cells were incubated with 40 nM DiOC₆ (Molecular probes, Eugene, Oreg.) for 30 min at 37° C. prior to FACS analysis.

Yeast Strains and Clonogenic Assays: 10⁴ cells/ml of Saccharomyces cerevisiae M 22-2-1 (genotype MATa ade2 leu2 lys2 his4 trp1 ura3 Canr, por1::LEU2, por2::TRP1; gift from Dr. M. Forte. Vollum Institute, Portland, Oreg.) (Blachly-Dyson, E. et al. J Biol Chem 268, 1835-41, 1993; Blachly-Dyson, E. et al. Mol Cell Biol 17, 5727-38, 1997), S. cerevisiae W301-1B control strain (MATa ade2, leu2, his3, trp1, ura3, can1), and JL1-3 (genotype like W301-1B, but aac1::LEU2 aac2::HIS3, aac3:: URA; gift from Dr. T. Drgon, National Institutes of Health, Bethesda, Md.) (Drgon, T. et al. FEBS Lett 289, 159-62, 1991), were pretreated with NFV followed by treatment with a Vpr-derived peptide (Genemed Synthesis Inc, San Francisco, Calif.) as described (Macreadie, I. G. et al. Mol Microbiol 19, 1185-92, 1996) or H₂O₂ (1 h, 28° C.). This was followed by plating on standard YPD agarose medium (200 yeasts/plate), and quantification of the percentage of surviving clones after 48 h of culture at 28° C.

Liposome technology: PTPC (1 mg/ml) was separated from rat brains and ANT (0.1 mg/ml) from rat heart mitochondria as previously described (Brenner, C. et al. Methods Enzymol 322, 243-52, 2000; Marzo, I. et al. Biomed Pharmacother 52, 248-51, 1998). Immediately after purification, pure proteins were reconstituted into proteoliposomes (phosphatidylcholin/cholesterol [5:1;w:w] for PTPC and phosphatidylcholin/cardiolipin [45:1; w:w] for ANT) (Belzacq, A. S. et al. Cancer Res 63, 541-6, 2003). After extensive dialysis to eliminate the surfactants, proteoliposomes were loaded with 4-methyllumbelliferylphosphate (4-MUP) in 10 mM KCl, 10 mM Hepes, 125 mM saccharose (pH 7.4), by sonication (25%, 22 sec on ice, Misonix 550), washed on Sephadex PD-10 columns (Amersham, France) and dispended in 96-well microtiter plates (Belzacq, A. S. et al. Cancer Res 63, 541-6, 2003). 25 μl of proteoliposomes were incubated with the indicated agents (30 min for NFV and 60 min for ATR and Vpr52-96 at RT). External 4-MUP was then converted in 4-MU by the addition of alkaline phosphatase in the presence of MgCl₂ for 15 min at 37° C. The release of 4-MU was measured by spectrofluorimetry (excitation wave length 360 nm; emission wave length 450 nm). The maximum 4-MUP release was determined by adding 5% Triton X-100 to proteoliposomes. The percentage of 4-MUP release induced by treatment of liposomes by ATR was determined as [(ATR−treated liposomes fluorescence−untreated liposomes fluorescence)/(TX-100-treated liposomes fluorescence−untreated liposomes fluorescence)]×100. The maximal fluorescence induced by 800 μM ATR was then identified as 100% 4-MUP release and the fluorescence induced by the treatment of liposomes by another product or another dose was calculated as a percentage of ATR-induced 4-MUP release.

Results

To assess the putative antiapoptotic effects of PI in vivo, we used three distinct and validated models of apoptotic pathology.

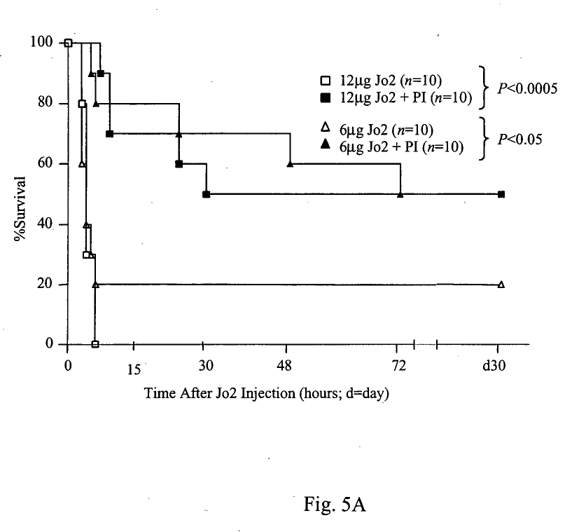

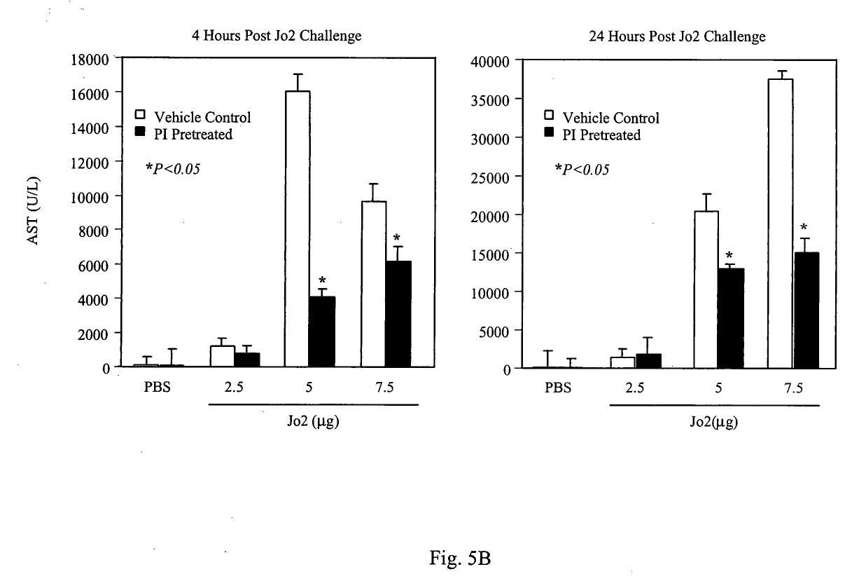

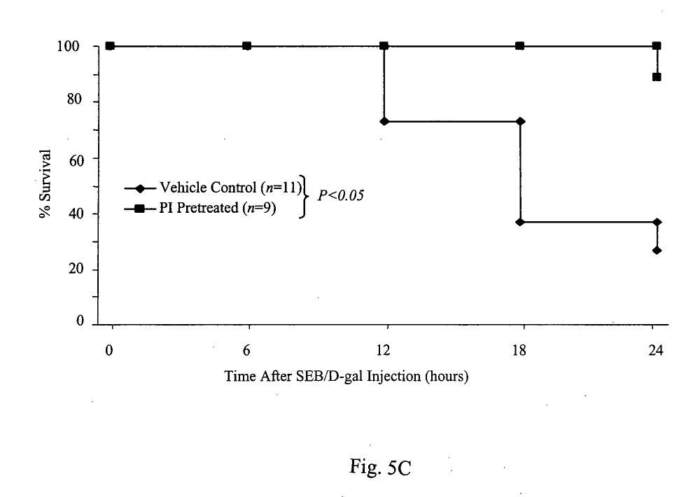

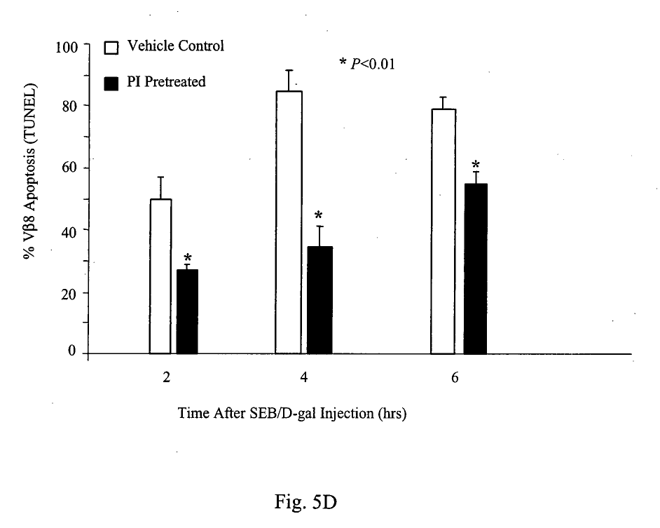

First, we evaluated the impact of PI treatment in CD95/Fas induced hepatic failure. This model has been previously used to evaluate antiapoptotic strategies including IL-6, and caspase 8 and Fas siRNA amongst others (Zender, L. et al. Proc Natl Acad Sci USA 100, 7797-802, 2003; Song, E. et al. Nat Med 9, 347-51, 2003; Kovalovich, K. et al. J Biol Chem 276, 26605-13, 2001). Mice received PI or control pretreatment for 24 hours, and then were challenged with 6 or 12 μg of intravenous (IV) Jo-2 anti-Fas antibody. Control animals developed shock and died in a dose responsive manner to Jo-2. In contrast, PI pretreated groups had superior survival when compared to mice receiving vehicle control (FIG. 5A). Importantly, all mice which died did so within 72 hours indicating that PI truly prevent rather than delay Jo-2 induced death. In parallel experiments, groups of ten mice each received 2.5, 5, or 7.5 μg of IV Jo-2 with or without PI pretreatment. Mice were sacrificed at 4 or 24 hours and analyzed for serum biochemistries, histology, or TUNEL. Serum glucose, BUN, creatinine, phosphorus, total protein, albumin, globulin, bilirubin, and cholesterol were similar between groups. However, PI pretreated mice had attenuated elevations in serum AST levels at both 4 and 24 hours compared to control mice (P<0.03) (FIG. 5B), reduced evidence of hepatitis at 48 hours as seen by H&E histology, and a reduction in TUNEL positive hepatocytes from a mean of 50%, to 15% (P=0.002). Further, hepatic histology at day 30 remained preserved in surviving PI treated mice.

To assess whether PI protection was applicable to other apoptotic processes, mice were treated with the bacterial superantigen Staphylococcal enterotoxin B (SEB) in the presence or absence of PI. Systemic treatment with SEB results in the selective apoptosis of SEB reactive T cells (which express the T cell receptor V-β8 chain) and shock. When co-administered with D-galactosamine (D-Gal), SEB injection also results in death (Miethke, T. et al. J Exp Med 175, 91-8, 1992; Aoki, Y. et al. Blood 86, 1420-7, 1995). Parallel groups of mice were pretreated for 24 hours with PI or control, followed by 20 mg of D-gal and 6.5 μg of SEB. PI treated mice had improved survival compared to controls (89%, n=9 vs 27%, n=11; P=0.0066) (FIG. 5C), as well as reduced V-β8 T cell apoptosis (FIG. 5D).

Given the profound reductions in target cell apoptosis and improvements in survival afforded by PI in the Jo-2 and SEB models, we finally assessed the impact of PI on neuronal apoptosis induced by two vessel cerebral occlusion.

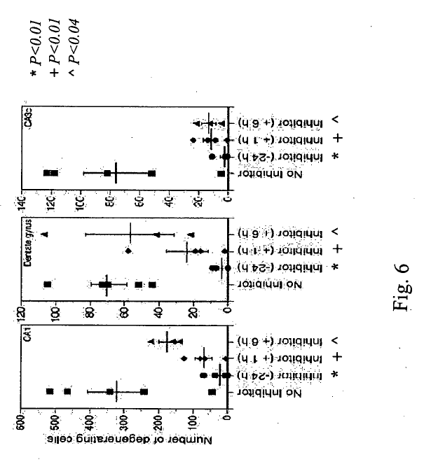

Using eosinophilic cytoplasm and hyperchromatic nuclei as a measure of neurodegeneration, control mice (that did not undergo carotid occlusion), had a mean of 7.8, 2.8, and 8.8 eosinophilic neurons per field in the CA-1, CA3c and dentate regions, respectively (FIG. 6) (n=5). Mice receiving vehicle control gavage followed by cerebral occlusion had significant neurodegeneration in the CA1 region (mean 322.2 cells per field, n=5, P<0.001), CA3c pyramidal cells (mean 69, n=5, P<0.001), and in granule cells of the dentate gyrus (mean 76, n=5, P<0.001) (FIG. 6). By comparison, administration of PI prior to ischemia resulted in substantial reduction in cell death (FIG. 6) in the CA1 (P=0.005), CA3c (P=0.015) and dentate regions (P=0.004) (FIG. 6). We next assessed the impact of PI treatment given after the ischemic insult. When PI treatment was initiated 1 hour following occlusion, and continued for 24 hours (n=4), the number of eosinophillic cells in the CA1, CA3c and granule cell layers was reduced by 78% (P=0.008), 84% (P=0.016) and 65% (P=0.008), respectively, compared to controls (FIG. 6). When PI therapy was initiated 6 hours after occlusion (n=3), the number of neurodegenerating cells in the CA1, CA3c, and granule cell layers was reduced by 45% (P=0.036), 82% (P=0.042), and 17% (P=0.036), respectively, compared to controls (FIG. 6).

In these experiments, vehicle treated animals exhibited numerous TUNEL-positive (mean >500) neurons in the pyramidal cell fields of the hippocampus and striatum when analyzed 48 hours post occlusion. In comparison, mice who received PI prior to carotid occlusion had undetectable or rare TUNEL-positive cells within the hippocampus (mean=27, P<0.01) and striatum (mean=17, P<0.01). When PI therapy was initiated within 1 hour of ischemia, there was no TUNEL staining within the hippocampus and the striatum. In mice that received their first dose of PI 6 hours following cerebral occlusion, the level of TUNEL-positive cells was reduced compared to controls, and was limited to the hippocampus (mean=126, P<0.01). P85 PARP is the apoptotic cleavage product of full-length PARP produced specifically by active caspase 3. P85 PARP staining was used as an independent measure of apoptosis, and confirmed the TUNEL results.

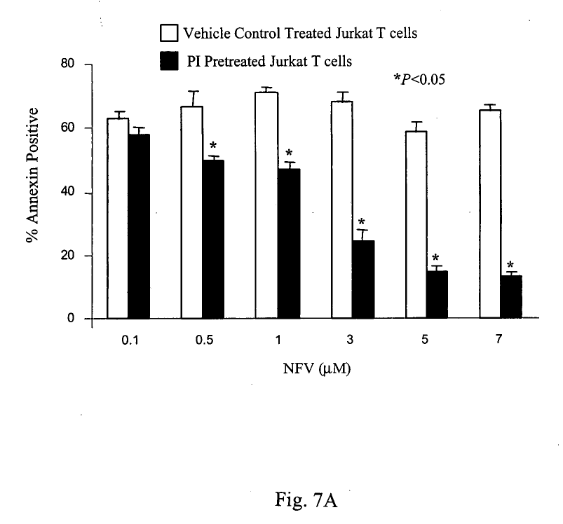

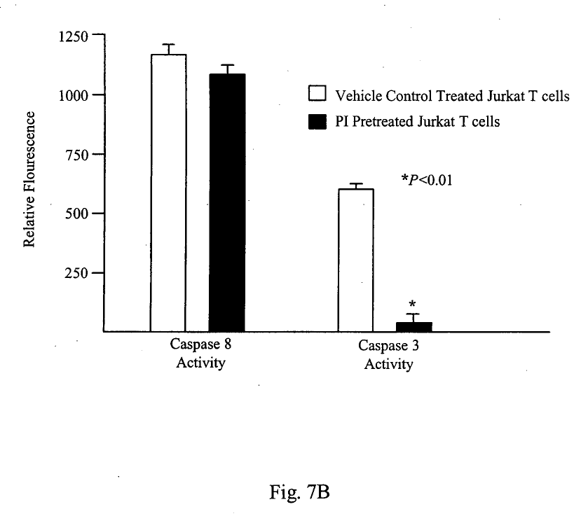

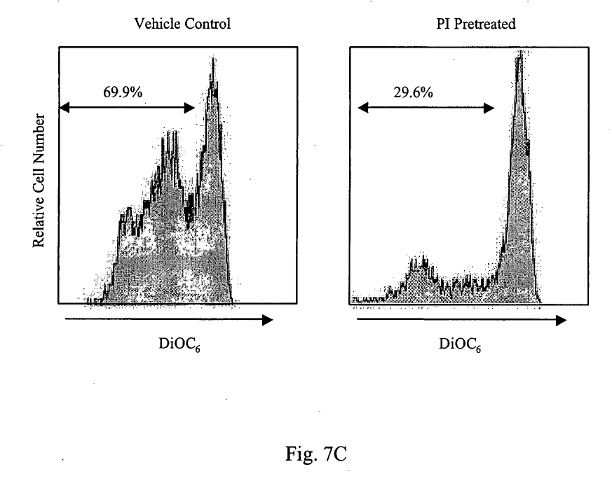

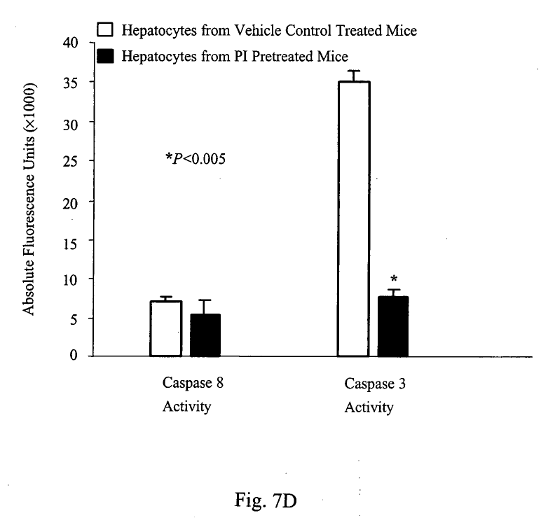

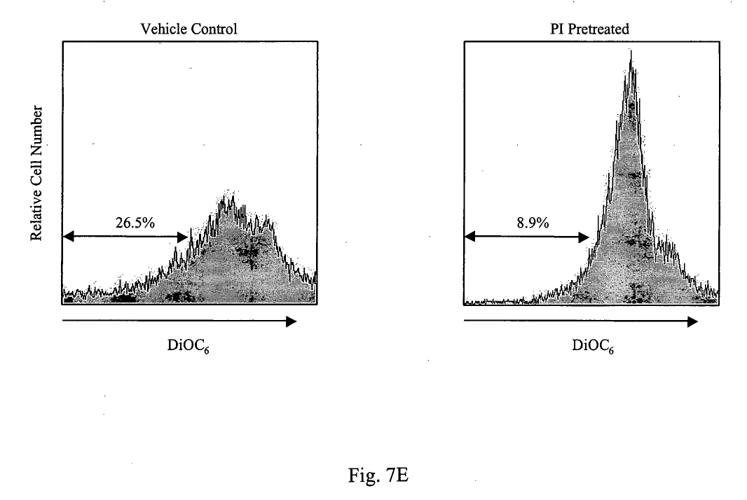

A variety of mechanisms have been proposed to account for the antiapoptotic effects of PI in vitro (reviewed in Phenix, B. N. et al. Apoptosis 7, 295-312, 2002). We opted to analyze the mechanism of PI inhibition of apoptosis in vivo, using the well characterized Fas signaling pathway. Jurkat T cells stimulated in vitro with CH-11 anti-Fas antibody were compared to hepatocytes isolated from mice treated in vivo with IV Jo-2 anti-Fas antibody for events of the Fas signaling cascade. In vitro, pretreatment of Jurkat T cells with nelfinavir (NFV) followed by CH-11 anti-CD95 antibody resulted in a dose-dependent reduction in apoptosis (FIG. 7A). Moreover, CH-11 treatment caused procaspase 8 cleavage and activity, Bid cleavage, loss of mitochondrial membrane potential (ΔΨₘ), cytochrome c release, procaspase 9 cleavages, and procaspase 3 cleavage and activity (see e.g., FIG. 7B, C). NFV pretreated Jurkat T cells had similar procaspase 8 cleavage and activity, and Bid cleavage as seen in vehicle control cells. However, the mitochondrial and post mitochondrial events Of ΔΨₘ, (as measured by loss of DiOC₆ retention), cytochrome c release, caspase 9, and caspase 3 cleavage and activity all reduced by NFV pretreatment (see e.g., FIG. 7B, C). Such data suggest that in vitro PI treatment blocks Fas induced apoptosis at the level of mitochondrial Δ™ₘ. Similar analyses were performed ex vivo on hepatocytes isolated from mice pretreated with either PI or vehicle control, and subsequently challenged with IV Jo-2. Similar to CH-11 treated Jurkat T cells, caspase 8 activation was unaltered by PI pretreatment, whereas caspase 3 activity was significantly (P<0.005) inhibited by PI pretreatment (FIG. 7D). Additionally, the ΔΨₘ loss present in vehicle control hepatocytes as inhibited in hepatocytes from mice who received NFV pretreatment (FIG. 7E). Thus, in both Jurkat T cells treated in vitro and hepatocytes treated in vivo with Fas, PI treatment inhibits mitochondrial ΔΨₘ and consequently the post-mitochondrial apoptosis signaling events.

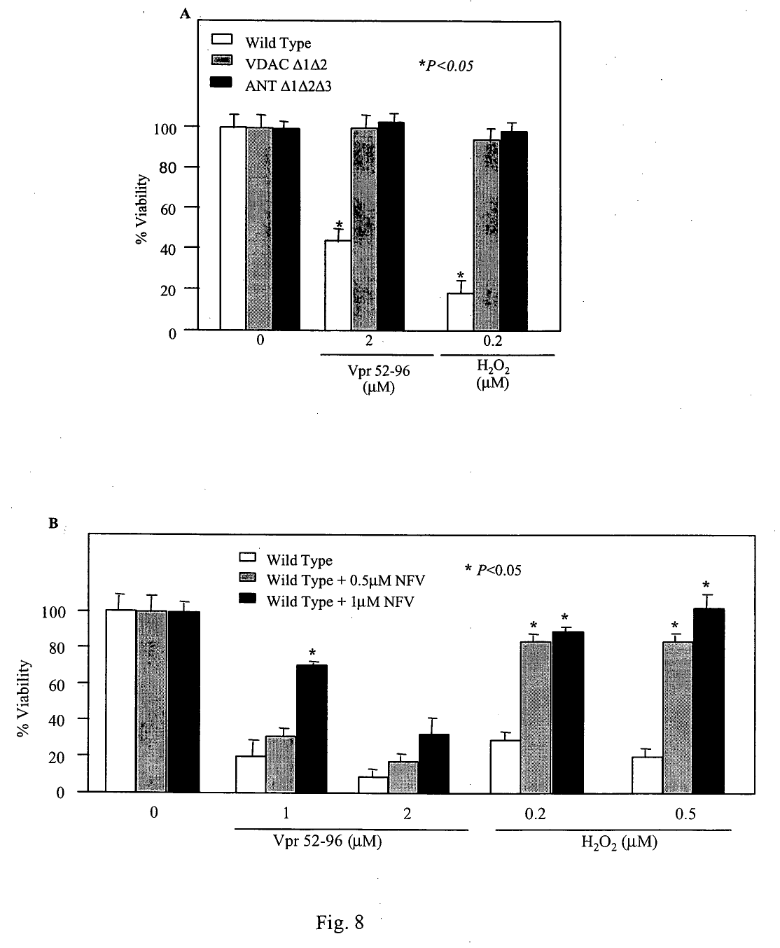

Mitochondria are central regulators of apoptotic signaling pathways in many forms of apoptosis (Kroemer, G. & Reed, J. C. Nat Med 6, 513-9, 2000). Following an appropriate apoptotic stimulus, loss of ΔΨₘ occurs, which is coincident with the opening of the mitochondrial permeability transition pore complex (PTPC). These events allow release of cytochrome c and Apaf-1 into the cytosolic compartment, where they complex with procaspase 9 to form the apoptosome, resulting in procaspase 9 activation which in turn activates the downstream effector caspase 3. A key regulator of this process therefore is the mitochondrial PTPC, which is composed of the peripheral benzodiazepine receptor (PBR), adenine nucleotide translocator (ANT), voltage dependent anion channel (VDAC), as well as other proteins. Given that our data in vitro and in vivo suggest that PI inhibit apoptosis via an effect on mitochondria, we questioned whether these key mitochondrial regulators of apoptosis were required to mediate this effect. For this, a yeast model of apoptosis was used involving wild type yeast, or yeast deficient of both isoforms of VDAC or all three isoforms of ANT. Previously, the ability of HIV Vpr to initiate apoptosis has been localized to both VDAC and ANT (Jacotot, E. et al. J Exp Med 193, 509-19, 2001), and therefore we used this property of Vpr to stimulate apoptosis in the different yeast strains. Consistent with prior reports, Vpr peptides could induce yeast death only if both ANT and VDAC were present (FIG. 8A). Similarly, H₂O₂ could only induce apoptosis if both ANT and VDAC were present, consistent with the proposed interaction of VDAC with ANT to form the principle pore channel of the PTPC (Halestrap, A. P. & Brennerb, C. Curr Med Chem 10, 1507-25, 2003). Next we assessed the ability of NFV to inhibit apoptosis in wild type yeast following apoptosis stimulation using the ANT and VDAC dependent stimuli Vpr or H₂O₂. As with previous results, wild type yeast were exquisitely sensitive to both Vpr and H₂O₂ induced apoptosis. Moreover, apoptosis induced by both stimuli were inhibited in a dose responsive manner by NFV (FIG. 8B), suggesting that NFV requires function as ANT, VDAC, or both.

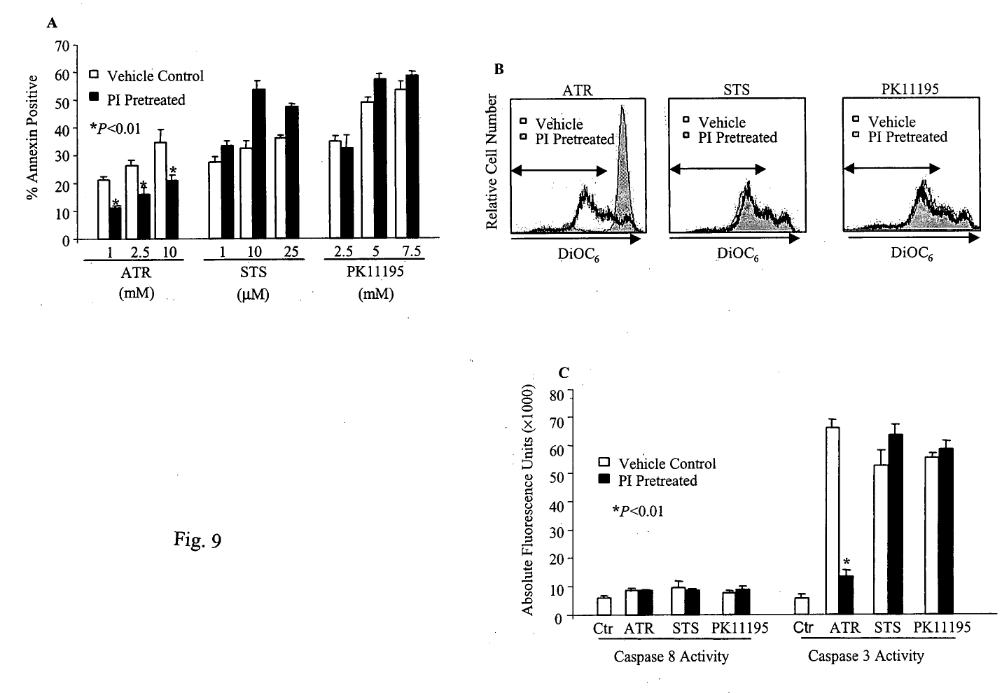

To further examine the involvement of VDAC and/or ANT in PI mediated apoptosis inhibition, we used chemical ligands which selectively interact with different components of the PTPC to result in its opening, and consequently loss of ΔΨₘ PK11195 is an agonist of the PBR (Decaudin, D. et al. Cancer Res 62, 1388-93, 2002; Hirsch, T. et al. Exp Cell Res 241, 426-34, 1998), atractyloside (ATR) is an agonist of ANT (Haouzi, D. et al. Apoptosis 7, 395-405, 2002), and staurosporine (STS) is an agonist of VDAC (Duan, S. et al. J Biol Chem 278, 1346-53, 2003). Jurkat T cells were pretreated with NFV or control and subsequently treated with these agonists and analyzed for apoptosis (FIG. 9A), ΔΨₘ (FIG. 9B), cytochrome c release, caspase 9 cleavage, caspase 3 cleavage, and PARP cleavage as well as caspase 3 activation (see e.g., FIG. 9C). Treatment with PK11195 or STS resulted in apoptosis, ΔΨₘ, cytochrome c release, caspase 9 cleavage, caspase 3 cleavages and activation of PARP cleavage that was not altered by PI, suggesting that PI do not alter PBR nor VDAC initiated loss of mitochondrial ΔΨₘ or the postmitochondrial events of apoptosis. Repeating these experiments with the ANT specific agonist ATR, caused loss of ΔΨₘ, cytochrome c release, caspase 9 cleavage, caspase 3 cleavage, PARP cleavage, and caspase 3 activation that were inhibited by PI (see e.g., FIG. 9A-C). These differential results with selective PTPC agonists suggest that NFV acts as an inhibitor of ANT dependent PTPC opening.

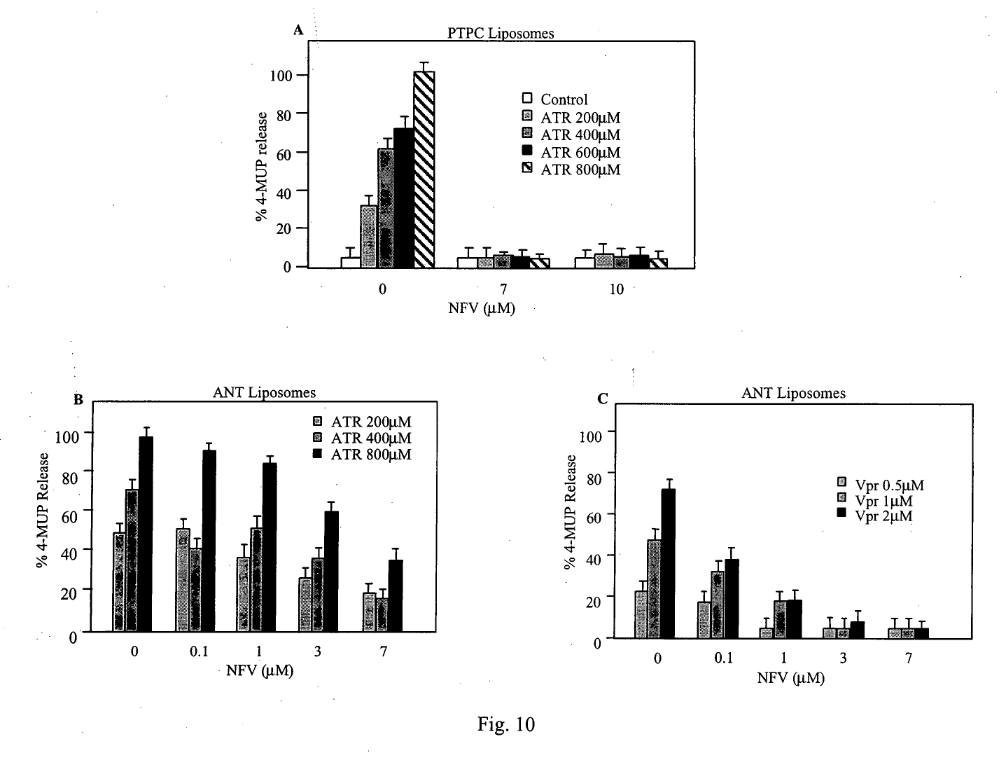

To confirm that PI effects are specific to ANT, we used proteoliposomes reconstituted with either PTPC or ANT alone. These liposomes contain a fluorescent dye which is released following pore opening (Brenner, C. et al. Methods Enzymol 322, 243-52, 2000). Consistent with our cellular data, treatment of PTPC liposomes with ATR results in significant fluorescence release that is progressively inhibited by increasing doses of NFV (FIG. 10A). We next assessed whether NFV could act directly upon ANT to inhibit its pore function. ANT liposomes were constructed and treated with two distinct ANT ligands, ATR (FIG. 10B) and Vpr (FIG. 10C). In both instances, each ligand resulted in a dose dependent release of fluorescence from the ANT liposomes that in turn was inhibited (p<0.05) in a dose responsive manner by NFV.

### Example 4

### Treating Multiple Sclerosis with HIV Proteins Inhibitors

We evaluated the effect of PI on Theilers virus induced mouse multiple sclerosis model. In this model, Theilers virus induces neuronal apoptosis and subsequent demyelination.

We treated 12 SLJ mice (6 in control group and 6 in PI-treated group) with either 125 mg/Kg NFV and 13 mg/Kg RIT or control vehicles every 8 hours for 48 hours, followed by Theilers virus infection. We observed that the PI treated mice had virtually no apoptotic neurons, but the control mice had many apoptotic neurons (p<0.05). In parallel experiments, Theilers virus was used to infect cells in vitro, and viral replication was quantified. PI did not impact viral replication.

### Example 5

### Treating Anthrax with HIV Proteins Inhibitors

The toxin from anthrax can induce apoptosis of cells, and this can contribute to the toxicity of anthrax. To test the effect of HIV PI on anthrax, we treated mouse monocytic cells (RAW264.7 cells) with 0, 1, 3, 5 or 7 μM NFV, followed 12 hours later by treatment with PA and LT (two components of anthrax toxin) at doses we had previously determined to induce apoptosis. In the control cells, there was up to 60% apoptosis, which was significantly inhibited in a dose responsive manner by NFV.

### Example 6

### Apoptosis Assays

There are numerous assays available to measure apoptosis known to one of skill in the art, which are useful in assessing the potential use of PI for the treatment of apoptotic conditions and indications. Some of the commonly used assays and experiments to evaluate apoptosis are set out below.

Nucleic Acid Staining: The characteristic breakdown of the nucleus during apoptosis comprises collapse and fragmentation of the chromatin, degradation of the nuclear envelope and nuclear blebbing, resulting in the formation of micronuclei. Therefore, nucleic acid stains are useful tools for identifying even low numbers of apoptotic cells in cell populations. Several nucleic acid stains may be used to detect apoptotic cells by fluorescence imaging or flow cytometry.

DNA Ladder: DNA fragmentation is detected in vitro using electrophoresis. DNA extracted from apoptotic cells, separated by gel electrophoresis, and stained with ethidium bromide reveals a characteristic ladder pattern of low molecular weight DNA fragments. Ethidium bromide may be used in a dot-blot assay to detect apoptotic DNA fragments. Ultrasensitive SYBR Green I nucleic acid stain and SYBR DX DNA blot stain also allow the detection of even fewer apoptotic cells in these applications.

Hoechst and Propidium Iodide Staining: The DNA stain, Hoechst 33342, is readily taken up by cells during the initial stages of apoptosis, whereas cell-impermeant dyes such as propidium iodide and ethidium bromide are excluded. Later stages of apoptosis are accompanied by an increase in membrane permeability, which allows propidium iodide to enter cells. Thus, a combination of Hoechst 33342 and propidium iodide is used for simultaneous flow cytometric and fluorescence imaging analysis of the stages of apoptosis and cell-cycle distribution. Hoechst 33342, which selectively stains nuclei of apoptotic cells blue fluorescent, is also used in combination with calcein AM, which labels all cells that have intact membranes—even apoptotic cells—green fluorescent. Presumably the dead-cell population is selectively detected using propidium iodide to make this a three-color assay.

TUNEL: Apoptotic cells also are identified by the use of the TUNEL assay (Intergen ApopTag kit, Intergen Company, Purchase, N.Y.). In the TUNEL assay, the enzyme terminal deoxynucleotidyl transferase labels 3′-OH DNA ends, generated during apoptosis, with biotinylated nucleotides. The latter are then detected by immunoperoxidase staining.

APO-BrdU TUNEL Assay: Because DNA fragmentation is one of the most reliable methods for detecting apoptosis, the APO-BrdU TUNEL Assay is also used to label and detect DNA strand breaks of apoptotic cells. When DNA strands are cleaved or nicked by nucleases, a large number of 3′-hydroxyl ends are exposed. In the APO-BrdU assay, these ends are labeled with BrdUTP and terminal deoxynucleotidyl transferase (TdT) using the TUNEL technique. Once incorporated into the DNA, BrdU is detected using an Alexa Fluor 488 dye-labeled anti-BrdU monoclonal antibody. Propidium iodide is used in this procedure to determine total cellular DNA content.

Caspase Activation: Caspase activation, one measure of activation of an apoptotic pathway, may be measured using a blue fluorescent substrate in the caspase assay (Promega, Madison, Wis.). Cleavage of the substrate by caspase 3 releases a fluorochrome, which then fluoresces green. This assay may be used to monitor activation of many caspases other than caspase 3 alone.

Annexin V: One of the early events of apoptosis is the loss of membrane asymmetry of phospholipids. At this early stage, the plasma membrane stays intact, but phosphatidylserine, normally located in the inner leaflet of the membrane, redistributes and appears in the outer leaflet. Annexins are a family of anticoagulant proteins that bind to phospholipid membranes in the presence of Ca²⁺. Annexin V binds specifically to phosphotidylserine (PS) on apoptotic cell surfaces and is used as a marker for apoptosis. In normal viable cells, PS is located on the cytoplasmic surface of the cell membrane. However, in apoptotic cells, PS is translocated from the inner to the outer leaflet of the plasma membrane, where it is associated with lipid “rafts”—regions of the plasma membrane that are insoluble in detergents, high in cholesterol and sphingolipids, that sequester glycosylphosphatidylinositol (GPI)-linked proteins and tyrosine-phosphorylated proteins and that seem to be involved in signal transduction. Highly fluorescent annexin V conjugates provide quick and reliable detection methods for studying the externalization of PS, one of the earliest indicators of apoptosis.

The present invention is not intended to be limited to the foregoing example, but to encompass all such modifications and variations as come within the scope of the appended claims.

1. A method of reducing or inhibiting apoptosis in a yeast cell or in a cell or tissue of a mammal wherein said mammal is not infected with HIV, the method comprising the step of exposing the yeast cell or the mammalian cell or tissue to an amount of one or more HIV protease inhibitors that is effective to reduce or inhibit apoptosis.

2. The method of claim 1, wherein an amount of one or more HIV protease inhibitors effective to reduce or inhibit apoptosis in the mammalian cell or tissue is administered to the mammal.

3. The method of claim 2, wherein the cell of the mammal is selected from the group consisting of hepatocytes, lymphocytes, neuronal cells, intestinal epithelial cells and cardiomyocytes.

4. The method of claim 2, wherein the mammal selected from the group consisting of a human, a dog, a cat, a rat, a mouse, a rabbit, a horse, a pig, a sheep, and a cattle.

5. The method of claim 4, wherein the mammal is a human.

6. The method of claim 1, wherein the one or more HIV protease inhibitors are selected from the group consisting of saquinavir, ritonavir, lopinavir, indinavir, VX-478, nelfinavir, KNI-272, CGP-61755, U-103017, cyclic protease inhibitors, and analogs, mimetics and derivatives thereof.

7. The method of claim 6, wherein a combination of nelfinavir and ritonavir is administered.

8. The method of claim 2, wherein the HIV protease inhibitor is administered orally.

9. The method of claim 2, wherein the HIV protease inhibitor is administered intravenously or subcutaneously.

10. A method of preventing or treating an apoptosis-related disease or condition in a mammal, wherein said mammal is not infected with HIV, the method comprising the step of administering to said mammal a therapeutically effective amount of one or more HIV protease inhibitors.

11. The method of claim 10, wherein the method is used for treating an apoptosis-related disease or condition.

12. The method of claim 10, wherein the method is used for preventing an apoptosis-related disease or condition and further comprising the step of identifying a mammal that is at risk of developing an apoptosis-related disease or condition.

13. The method of claim 10, wherein the apoptosis-related disease is selected from the group consisting of sepsis, septic shock, stroke, hepatitis, multiple sclerosis, amyotrophic lateral sclerosis, and anthrax toxin or enterotoxin mediated diseases or conditions.

14. The method of claim 10, wherein the mammal selected from the group consisting of a human, a dog, a cat, a rat, a mouse, a rabbit, a horse, a pig, a sheep, and a cattle.

15. The method of claim 14, wherein the mammal is a human.

16. The method of claim 10, wherein the one or more HIV protease inhibitors are selected from the group consisting of saquinavir, ritonavir, lopinavir, indinavir, VX-478, nelfinavir, KNI-272, CGP-61755, U-103017, cyclic protease inhibitors, and analogs, mimetics and derivatives thereof.

17. The method of claim 10, wherein a combination of nelfinavir and ritonavir is administered.

18. The method of claim 10, wherein the HIV protease inhibitor is administered orally.

19. The method of claim 10, wherein the HIV protease inhibitor is administered intravenously or subcutaneously.

20. A method of reducing or inhibiting apoptosis in a yeast cell or a cell or tissue of a mammal wherein said mammal is not infected with HIV, the method comprising the step of reducing or inhibiting at least one of mitochondrial adenine nucleotide translocator (ANT) dependent permeability transition pore complex (PTPC) opening and mitochondrial cytochrome c release in the yeast cell or the mammalian cell or tissue.

21. The method of claim 20, wherein the method is for reducing or inhibiting apoptosis in a cell or tissue of a mammal

22. The method of claim 21, wherein the cell of the mammal is selected from the group consisting of hepatocytes, lymphocytes, neuronal cells, intestinal epithelial cells and cardiomyocytes.

23. The method of claim 21, wherein the mammal selected from the group consisting of a human, a dog, a cat, a rat, a mouse, a rabbit, a horse, a pig, a sheep, and a cattle.

24. The method of claim 23, wherein the mammal is a human.

25. A method of preventing or treating an apoptosis-related disease or condition in a mammal, wherein said mammal is not infected with HIV, the method comprising the step of reducing or inhibiting at least one of mitochondrial adenine nucleotide translocator (ANT) dependent permeability transition pore complex (PTPC) opening and mitochondrial cytochrome c release in target cells or tissue.

26. The method of claim 25, wherein the method is used for treating an apoptosis-related disease or condition.

27. The method of claim 25, wherein the method is used for preventing an apoptosis-related disease or condition and further comprising the step of identifying a mammal that is at risk of developing an apoptosis-related disease or condition.

28. The method of claim 25, wherein the apoptosis-related disease is selected from the group consisting of sepsis, septic shock, stroke, hepatitis, multiple sclerosis, amyotrophic lateral sclerosis, and anthrax toxin or enterotoxin mediated diseases or conditions.

29. The method of claim 25, wherein the mammal selected from the group consisting of a human, a dog, a cat, a rat, a mouse, a rabbit, a horse, a pig, a sheep, and a cattle.

30. The method of claim 29, wherein the mammal is a human.

31. A pharmaceutical kit comprising an HIV protease inhibitor and instruction on how to use the inhibitor to prevent or treat an apoptosis-related disease or condition.
